(19) United States
(12) Patent Application Publication
(10) Pub. No.: US 2014/0275063 A1
(43) Pub. Date: Sep. 18, 2014

(54) NEW ENZYME INHIBITOR COMPOUNDS

(71) Applicants: Allison Carley, London (GB); Iain Simpson, London (GB)

(72) Inventors: Allison Carley, London (GB); Iain Simpson, London (GB)

(73) Assignee: PROXIMAGEN LIMITED, London (GB)

(21) Appl. No.: 14/344,436

(22) PCT Filed: Sep. 13, 2012
(86) PCT No.: PCT/GB2012/052265

§ 371 Date: May 2, 2014

(30) Foreign Application Priority Data
Sep. 14, 2011 (GB) 1115853.2

(51) Int. Cl. C07D 471/04

(52) U.S. Cl. CPC C07D 471/04
USPC 514/232.5; 546/113; 514/300; 544/362; 514/253.04; 544/127; 514/234.5; 544/121; 514/234.2; 546/119; 514/303

(57) ABSTRACT

Compounds of formula (I) are inhibitors of Semicarbazide-sensitive amine oxidase

R¹—X—R² (I)

wherein R¹, X and R² are as defined in the claims.

### FIELD OF THE INVENTION

The present invention relates to compounds which are inhibitors of SSAO activity. The invention also relates to pharmaceutical compositions comprising these compounds and to the use of these compounds in the treatment or prevention of medical conditions wherein inhibition of SSAO activity is beneficial, such as inflammatory diseases, immune disorders and the inhibition of tumour growth.

### BACKGROUND ART

Semicarbazide-sensitive amine oxidase (SSAO) activity is an enzyme activity expressed by Vascular Adhesion Protein-1 (VAP-1) or Amine Oxidase, Copper Containing 3 (AOC3), belongs to the copper-containing amine oxidase family of enzymes (EC.1.4.3.6). Therefore inhibitors of the SSAO enzyme may also modulate the biological functions of the VAP-1 protein. Members of this enzyme family are sensitive to inhibition by semicarbazide and utilize cupric ion and protein-derived topa quinone (TPQ) cofactor in the oxidative deamination of primary amines to aldehydes, hydrogen peroxide, and ammonia according to the following reaction:

R—CH₂—NH₂+O₂→R—CHO+H₂O₂+NH₃

Known substrates for human SSAO include endogenous methylamine and aminoacetone as well as some xenobiotic amines such as benzylamine [Lyles, Int. J. Biochem. Cell Biol. 1996, 28, 259-274; Klinman, Biochim. Biophys. Acta 2003, 1647(1-2), 131-137; Mátyus et al., Curr. Med. Chem. 2004, 11(10), 1285-1298; O'Sullivan et al., Neurotoxicology 2004, 25(1-2), 303-315]. In analogy with other copper-containing amine oxidases, DNA-sequence analysis and structure determination suggest that the tissue-bound human SSAO is a homodimeric glycoprotein consisting of two 90-100 kDa subunits anchored to the plasma membrane by a single N-terminal membrane spanning domain [Morris et al., J. Biol. Chem. 1997, 272, 9388-9392; Smith et al., J. Exp. Med. 1998, 188, 17-27; Airenne et al., Protein Science 2005, 14, 1964-1974; Jakobsson et al., Acta Crystallogr. D Biol. Crystallogr. 2005, 61(Pt 11), 1550-1562].

SSAO activity has been found in a variety of tissues including vascular and non-vascular smooth muscle tissue, endothelium, and adipose tissue [Lewinsohn, Braz. J. Med. Biol. Res. 1984, 17, 223-256; Nakos & Gossrau, Folia Histochem. Cytobiol. 1994, 32, 3-10; Yu et al., Biochem. Pharmacol. 1994, 47, 1055-1059; Castillo et al., Neurochem. Int. 1998, 33, 415-423; Lyles & Pino, J. Neural. Transm. Suppl. 1998, 52, 239-250; Jaakkola et al., Am. J. Pathol. 1999, 155, 1953-1965; Morin et al., J. Pharmacol. Exp. Ther. 2001, 297, 563-572; Salmi & Jalkanen, Trends Immunol. 2001, 22, 211-216]. In addition, SSAO protein is found in blood plasma and this soluble form appears to have similar properties as the tissue-bound form [Yu et al., Biochem. Pharmacol. 1994, 47, 1055-1059; Kurkijärvi et al., J. Immunol. 1998, 161, 1549-1557]. It has recently been shown that circulating human and rodent SSAO originates from the tissue-bound form [Göktürk et al., Am. J. Pathol. 2003, 163(5), 1921-1928; Abella et al., Diabetologia 2004, 47(3), 429-438; Stolen et al., Circ. Res. 2004, 95(1), 50-57], whereas in other mammals the plasma/serum SSAO is also encoded by a separate gene called AOC4 [Schwelberger, J. Neural. Transm. 2007, 114(6), 757-762].

The precise physiological role of this abundant enzyme has yet to be fully determined, but it appears that SSAO and its reaction products may have several functions in cell signalling and regulation. For example, recent findings suggest that SSAO plays a role in both GLUT4-mediated glucose uptake [Enrique-Tarancon et al., J. Biol. Chem. 1998, 273, 8025-8032; Morin et al., J. Pharmacol. Exp. Ther. 2001, 297, 563-572] and adipocyte differentiation [Fontana et al., Biochem. J. 2001, 356, 769-777; Mercier et al., Biochem. J. 2001, 358, 335-342]. In addition, SSAO has been shown to be involved in inflammatory processes where it acts as an adhesion protein for leukocytes [Salmi & Jalkanen, Trends Immunol. 2001, 22, 211-216; Salmi & Jalkanen, in “Adhesion Molecules: Functions and Inhibition” K. Ley (Ed.), 2007, pp. 237-251], and might also play a role in connective tissue matrix development and maintenance [Langford et al., Cardiovasc. Toxicol. 2002, 2(2), 141-150; Gökürk et al., Am. J. Pathol. 2003, 163(5), 1921-1928]. Moreover, a link between SSAO and angiogenesis has recently been discovered [Noda et al., FASEB J. 2008, 22(8), 2928-2935], and based on this link it is expected that inhibitors of SSAO have an anti-angiogenic effect.

Several studies in humans have demonstrated that SSAO activity in blood plasma is elevated in conditions such as congestive heart failure, diabetes mellitus, Alzheimer's disease, and inflammation [Lewinsohn, Braz. J. Med. Biol. Res. 1984, 17, 223-256; Boomsma et al., Cardiovasc. Res. 1997, 33, 387-391; Ekblom, Pharmacol. Res. 1998, 37, 87-92; Kurkijärvi et al., J. Immunol. 1998, 161, 1549-1557; Boomsma et al., Diabetologia 1999, 42, 233-237; Meszaros et al., Eur. J. Drug Metab. Pharmacokinet. 1999, 24, 299-302; Yu et al., Biochim. Biophys. Acta 2003, 1647(1-2), 193-199; Matyus et al., Curr. Med. Chem. 2004, 11(10), 1285-1298; O'Sullivan et al., Neurotoxicology 2004, 25(1-2), 303-315; del Mar Hernandez et al., Neurosci. Lett. 2005, 384(1-2), 183-187]. The mechanisms underlying these alterations of enzyme activity are not clear. It has been suggested that reactive aldehydes and hydrogen peroxide produced by endogenous amine oxidases contribute to the progression of cardiovascular diseases, diabetic complications and Alzheimer's disease [Callingham et al., Prog. Brain Res. 1995, 106, 305-321; Ekblom, Pharmacol. Res. 1998, 37, 87-92; Yu et al., Biochim. Biophys. Acta 2003, 1647(1-2), 193-199; Jiang et al., Neuropathol Appl Neurobiol. 2008, 34(2), 194-204]. Furthermore, the enzymatic activity of SSAO is involved in the leukocyte extravasation process at sites of inflammation where SSAO has been shown to be strongly expressed on the vascular endothelium [Salmi et al., Immunity 2001, 14(3), 265-276; Salmi & Jalkanen, in “Adhesion Molecules: Functions and Inhibition” K. Ley (Ed.), 2007, pp. 237-251]. Accordingly, inhibition of SSAO has been suggested to have a therapeutic value in the prevention of diabetic complications and in inflammatory diseases [Ekblom, Pharmacol. Res. 1998, 37, 87-92; Salmi et al., Immunity 2001, 14(3), 265-276; Salter-Cid et al., J. Pharmacol. Exp. Ther. 2005, 315(2), 553-562].

SSAO knockout animals are phenotypically overtly normal but exhibit a marked decrease in the inflammatory responses evoked in response to various inflammatory stimuli [Stolen et al., Immunity 2005, 22(1), 105-115]. In addition, antagonism of its function in wild type animals in multiple animal models of human disease (e.g. carrageenan-induced paw inflammation, oxazolone-induced colitis, lipopolysaccharide-induced lung inflammation, collagen-induced arthritis, endotoxin-induced uveitis) by the use of antibodies and/or small molecules has been shown to be protective in decreasing the leukocyte infiltration, reducing the severity of the disease phenotype and reducing levels of inflammatory cytokines and chemokines [Kirton et al., Eur. J. Immunol. 2005, 35(11), 3119-3130; Salter-Cid et al., J. Pharmacol. Exp. Ther. 2005, 315(2), 553-562; McDonald et al., Annual Reports in Medicinal Chemistry 2007, 42, 229-243; Salmi & Jalkanen, in “Adhesion Molecules: Functions and Inhibition” K. Ley (Ed.), 2007, pp. 237-251; Noda et al., FASEB J. 2008 22(4), 1094-1103; Noda et al., FASEB J. 2008, 22(8), 2928-2935]. This anti-inflammatory protection seems to be afforded across a wide range of inflammatory models all with independent causative mechanisms, rather than being restricted to one particular disease or disease model. This would suggest that SSAO may be a key nodal point for the regulation of the inflammatory response, and it is therefore likely that SSAO inhibitors will be effective anti-inflammatory drugs in a wide range of human diseases. VAP-1 has also been implicated in the progression and maintenance of fibrotic diseases including those of the liver and lung. Weston and Adams (J Neural Transm. 2011, 118(7), 1055-64) have summarised the experimental data implicating VAP-1 in liver fibrosis, and Weston et al (EASL Poster 2010) reported that blockade of VAP-1 accelerated the resolution of carbon tetrachloride induced fibrosis. In addition VAP-1 has been implicated in inflammation of the lung (e.g. Singh et al., 2003, Virchows Arch 442:491-495) suggesting that VAP-1 blockers would reduce lung inflammation and thus be of benefit to the treatment of cystic fibrosis by treating both the pro-fibrotic and pro-inflammatory aspects of the disease.

SSAO (VAP-1) is up regulated in gastric cancer and has been identified in the tumour vasculature of human melanoma, hepatoma and head and neck tumours (Yoong K F, McNab G, Hubscher S G, Adams D H. (1998), J Immunol 160, 3978-88; Irjala H, Salmi M, Alanen K, Gre nm an R, Jalkanen S (2001), Immunol. 166, 6937-6943; Forster-Horvath C, Dome B, Paku S, et al. (2004), Melanoma Res. 14, 135-40). One report (Marttila-lchihara F, Castermans K, Auvinen K, Oude Egbrink M G, Jalkanen S, Griffioen A W, Salmi M. (2010), J. Immunol. 184, 3164-3173) has shown that mice bearing enzymically inactive VAP-1 grow melanomas more slowly, and have reduced tumour blood vessel number and diameter. The reduced growth of these tumours was also reflected in the reduced (by 60-70%) infiltration of myeloid suppressor cells. Encouragingly VAP-1 deficiency had no effect on vessel or lymph formation in normal tissue.

Small molecules of different structural classes have previously been disclosed as SSAO inhibitors, for example in WO 0238153 (tetrahydroimidazo[4,5-c]pyridine derivatives), in WO 03006003 (2-indanylhydrazine derivatives), in WO 2005014530 (allylhydrazine and hydroxylamine (aminooxy) compounds) and in WO 2007120528 (allylamino compounds). Additional SSAO inhibitors are disclosed in PCT/EP2009062011 and PCT/EP2009062018.

The invention described here relates to a new class of SSAO inhibitors with biological, pharmacological, and pharmacokinetic characteristics that make them suitable for use as prophylactic or therapeutic agents in a wide range of human inflammatory diseases and immune disorders. This therapeutic capacity is designed to block SSAO enzyme action, reducing the levels of pro-inflammatory enzyme products (aldehydes, hydrogen peroxide and ammonia) whilst also decreasing the adhesive capacity of immune cells and correspondingly their activation and final extra-vasation. Diseases where such an activity is expected to be therapeutically beneficial include all diseases where immune cells play a prominent role in the initiation, maintenance or resolution of the pathology, such as multiple sclerosis, arthritis and vasculitis.

### DETAILED DESCRIPTION OF THE INVENTION

It has surprisingly been found that the compounds of formula (I) below are inhibitors of SSAO. They are therefore useful for the treatment or prevention of diseases in which inhibition of SSAO activity is beneficial, such as inflammation, inflammatory diseases, immune or autoimmune disorders, and inhibition of tumour growth.

According to the invention there is provided a compound of formula (I) or a pharmaceutically acceptable salt, or N-oxide thereof:

R¹—X—R² (I)

wherein
R¹ is phenyl or 6-membered heteroaryl, optionally substituted with one or more substituents selected from halogen, cyano, C₁₋₄-alkyl, halo-C₁₋₄-alkyl, C₁₋₄alkoxy-C₁₋₄alkyl, hydroxy-C₁₋₄-alkyl, cyano-C₁₋₄-alkyl, amino-C₁₋₄-alkyl, C₁₋₄-alkylamino-C₁₋₄-alkyl, di(C₁₋₄-alkyl)amino-C₁₋₄-alkyl, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵;

### R² is —B-Q-[R³]ₙ or —B—R³;

wherein n=1, 2, 3, or 4
B is a bond, O, NR⁴, —C(O)— or C₁₋₃-alkylene;
Q is saturated or partially unsaturated monocyclic 3-7 membered heterocyclic or C₃₋₇-cycloalkyl ring;
when R² is —B-Q-[R³]ₙ, R³ is independently selected from: 3-7 membered heterocyclyl-, 3-7 membered heterocyclyl-C₁₋₄-alkyl-, (3-7 membered heterocyclyl-C₁₋₄-alkyl)-amino-C₁₋₄-alkyl-, amino-C₁₋₄-alkoxy-C₁₋₄-alkyl-, (amino-C₁₋₄-alkyl)-amino-C₁₋₄-alkyl-, —C₁₋₄-alkyl-NR⁶C(O)OR⁵, —C₁₋₄-alkyl-NR⁶C(O)NR^{4A}R^{4B}, —C₁₋₄-alkyl-C(O)NR^{4A}R^{4B}, (3-7 membered heterocyclyl-C₁₋₄-alkyl)-C(O)—, —C₁₋₄-alkyl-C(O)OR⁵, —OC(O)R⁵, or
—C(O)NR^{9A}R^{9B} wherein R^{9A} and R^{9B} together with the nitrogen to which they are attached form a 3-7 membered cyclic amino group substituted with one or more substituents selected from: C₁₋₄-alkyl, C₁₋₄alkoxy-C₁₋₄alkyl-, C₃₋₇-cycloalkyl, or
—C(O)NR⁶R^{10B} wherein R^{10B} is:
(i) 3-7 membered heterocyclyl- or 3-7 membered heterocyclyl-C₁₋₄-alkyl-, or —C₁₋₄-alkyl-NR⁶C(O)R⁵; or
(ii) 5 or 6 membered heteroaryl-C₁₋₄-alkyl-, wherein the heteroaryl ring is optionally substituted with one or more substituents selected from halogen, cyano, C₁₋₄-alkyl, halo-C₁₋₄-alkyl, and wherein the C₁₋₄-alkyl part is optionally substituted by one or more C₁₋₄-alkyl- groups, or the C₁₋₄-alkyl part is substituted with two C₁₋₄-alkyl groups which, together with the carbon atom to which they are attached, join together to form a spiro 3-6 membered cycloalkyl ring; and wherein
when R² is —B—R³, R³ is —NR⁶R^{11B}, and R^{11B} is 3-7 membered heterocyclyl-C₁₋₄-alkyl-;
R^{4A}, R^{4B} and R⁵ are each independently selected from hydrogen, C₁₋₄-alkyl-, 3-7 membered heterocyclyl-C₁₋₄-alkyl-, amino-C₁₋₄-alkyl-, 3-7 membered heterocyclyl-, —C₁₋₄-alkyl-NR⁶C(O)OR⁶, C₃₋₇-cycloalkyl, or
R^{4A} and R^{4B} together with the nitrogen to which they are attached form a 3-7 membered cyclic amino group, optionally substituted by one or more substituents selected from: C₁₋₄-alkyl, —NR^{4A}R^{4B}; and wherein
unless otherwise specified, 3-7 membered heterocyclyl, or the heterocyclyl part of the 3-7 membered heterocyclyl-C₁₋₄-alkyl-, (3-7 membered heterocyclyl-C₁₋₄-alkyl)-amino-C₁₋₄-alkyl-, or (3-7 membered heterocyclyl-C₁₋₄-alkyl)-C(O)— group is optionally substituted with one or more substituents selected from oxo, C₁₋₄-alkyl-, —C(O)OR⁵, —C(O)R⁵, —C(O)NR^{4A}R^{4B}, —NR^{4A}R^{4B}, —C₁₋₄-alkyl-C(O)NR^{4A}R^{4B}, or C₁₋₄alkoxy-C₁₋₄alkyl; and
where present, the diradical —C₁₋₄-alkyl- group directly attached to Q is optionally substituted with one or more groups independently selected from halogen, amino, methoxy, hydroxyl; and wherein
R⁴ and R⁶ are each independently selected from hydrogen or C₁₋₄-alkyl; and
X is selected from the radicals of formulae (1-16) wherein the bond marked * is attached to R¹— and the bond marked ** is attached to —R²:

[chemical structure]

[chemical structure]

[chemical structure]

wherein Y is selected from hydrogen, hydroxyl, amino, —NHR⁶, —OCH₃;
Z is selected from hydrogen, fluorine, hydroxyl, C₁₋₄-alkoxy, halo-C₁₋₄-alkyl, CONH₂, cyano, SO₂NH₂, amino, —NHR⁶;
W is selected from H, C₁₋₄-alkyl, halo-C₁₋₄-alkyl,
PROVIDED THAT when R² is —B-Q-[R³]ₙ, and R³ is 3-7 membered heterocyclyl-, the R³ heterocyclic ring atom directly bonded to Q is not nitrogen.

In a related embodiment, the present invention makes available a compound of formula (I) or a pharmaceutically acceptable salt, or N-oxide thereof:

R¹—X—R² (I)

wherein
R¹ is phenyl or 6-membered heteroaryl, optionally substituted with one or more substituents selected from halogen, cyano, C₁₋₄-alkyl, halo-C₁₋₄-alkyl, C₁₋₄alkoxy-C₁₋₄alkyl, hydroxy-C₁₋₄-alkyl, cyano-C₁₋₄-alkyl, amino-C₁₋₄-alkyl, C₁₋₄-alkylamino-C₁₋₄-alkyl, di(C₁₋₄-alkylamino-C₁₋₄-alkyl, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵;

### R² is —B-Q-[R³]ₙ or —B—R³;

wherein n=1, 2, 3, or 4
B is a bond, O, NR⁴, —C(O)— or C₁₋₃-alkylene;
Q is saturated or partially unsaturated monocyclic 3-7 membered heterocyclic or C₃₋₇-cycloalkyl ring;
when R² is —B-Q-[R³]ₙ, R³ is independently selected from: 3-7 membered heterocyclyl-, 3-7 membered heterocyclyl-C₁₋₄-alkyl-, (3-7 membered heterocyclyl-C₁₋₄-alkyl)-amino-C₁₋₄-alkyl-, amino-C₁₋₄-alkoxy-C₁₋₄-alkyl-, (amino-C₁₋₄-alkyl)-amino-C₁₋₄-alkyl-, —C₁₋₄-alkyl-NR⁶C(O)OR⁵, —C₁₋₄-alkyl-NR⁶C(O)NR^{4A}R^{4B}, —C₁₋₄-alkyl-C(O)NR^{4A}R^{4B}, (3-7 membered heterocyclyl-C₁₋₄-alkyl)-C(O)—, —C₁₋₄-alkyl-C(O)OR⁵, —OC(O)R⁵, or
—C(O)NR^{9A}R^{9B} wherein R^{9A} and R^{9B} together with the nitrogen to which they are attached form a 3-7 membered cyclic amino group substituted with one or more substituents selected from: C₁₋₄-alkyl, C₁₋₄alkoxy-C₁₋₄alkyl-, C₃₋₇-cycloalkyl, or
—C(O)NR⁶R^{10B} wherein R^{10B} is 3-7 membered heterocyclyl- or 3-7 membered heterocyclyl-C₁₋₄-alkyl-, or —C₁₋₄-alkyl-NR⁶C(O)R⁵; or
when R² is —B—R³, R³ is —NR⁶R^{11B}, wherein R^{11B} is 3-7 membered heterocyclyl-C₁₋₄-alkyl-;
R^{4A}, R^{4B} and R⁵ are each independently selected from hydrogen, C₁₋₄-alkyl-, 3-7 membered heterocyclyl-C₁₋₄-alkyl-, amino-C₁₋₄-alkyl-, 3-7 membered heterocyclyl-, —C₁₋₄-alkyl-NR⁶C(O)OR⁵, C₃₋₇-cycloalkyl,
or R^{4A} and R^{4B} together with the nitrogen to which they are attached form a 3-7 membered cyclic amino group, optionally substituted by one or more substituents selected from: C₁₋₄-alkyl, —NR^{4A}R^{4B};
unless otherwise specified, 3-7 membered heterocyclyl, or the heterocyclyl part of the 3-7 membered heterocyclyl-C₁₋₄-alkyl-, (3-7 membered heterocyclyl-C₁₋₄-alkyl)-amino-C₁₋₄-alkyl-, or (3-7 membered heterocyclyl-C₁₋₄-alkyl)-C(O)— group is optionally substituted with one or more substituents selected from C₁₋₄-alkyl-, —C(O)OR⁵, —C(O)R⁵, —C(O)NR^{4A}R^{4B}, —NR^{4A}R^{4B}, —C₁₋₄-alkyl-C(O)NR^{4A}R^{4B}, or C₁₋₄alkoxy-C₁₋₄alkyl; and
where present, the diradical —C₁₋₄-alkyl- group directly attached to Q is optionally substituted with one or more groups independently selected from halogen, amino, methoxy, hydroxyl;
R⁴ and R⁶ are each independently selected from hydrogen or C₁₋₄-alkyl; and
X is selected from the radicals of formulae (1-16) wherein the bond marked * is attached to R¹— and the bond marked ** is attached to —R²:

[chemical structure]

[chemical structure]

[chemical structure]

wherein Y is selected from hydrogen, hydroxyl, amino, —NHR⁶, —OCH₃;
Z is selected from hydrogen, fluorine, hydroxyl, C₁₋₄-alkoxy, halo-C₁₋₄-alkyl, CONH₂, cyano, SO₂NH₂, amino, —NHR⁶;
W is selected from H, C₁₋₄alkyl, halo-C₁₋₄-alkyl,
PROVIDED THAT when R² is —B-Q-[R³]ₙ, and R³ is 3-7 membered heterocyclyl-, the heterocyclic ring atom directly bonded to Q is not nitrogen.

It is expected that compounds of the invention may be prepared in the form of hydrates, and solvates. Any reference herein, including the claims herein, to “compounds with which the invention is concerned” or “compounds of the invention” or “the present compounds”, and the like, includes reference to salts, hydrates, and solvates of such compounds. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Individual compounds of the invention may exist in an amorphous form and for several polymorphic forms and may be obtained in different crystal habits. Any reference herein, including the claims herein, to “compounds with which the invention is concerned” or “compounds of the invention” or “the present compounds”, and the like, includes reference to the compounds irrespective of amorphous or polymorphic form.

Since compounds of the invention have a nitrogen atom in an aromatic ring they may form N-oxides, and the invention includes compounds of the invention in their N-oxide form.

### DEFINITIONS

The following definitions shall apply throughout the specification and the appended claims, unless otherwise stated or indicated.

The term “C₁₋₄-alkyl” denotes a straight or branched alkyl group having from 1 to 4 carbon atoms. For parts of the range C₁₋₄-alkyl all subgroups thereof are contemplated such as C₁₋₃-alkyl, C₁₋₂-alkyl, C₂₋₄-alkyl, C₂₋₃-alkyl and C₃₋₄-alkyl. Examples of said C₁₋₄-alkyl include methyl, ethyl, n-propyl, isopropyl, n-butyl, isobutyl, sec-butyl and tert-butyl.

Unless otherwise specified, the term “C₃₋₇-cycloalkyl” refers to a monocyclic saturated or partially unsaturated hydrocarbon ring system having from 3 to 7 carbon atoms. Examples of said C₃₋₇-cycloalkyl include cyclopropyl, cyclobutyl, cyclopentyl, cyclohexyl, cyclohexenyl, cycloheptyl, and cycloheptenyl. For parts of the range “C₃₋₇-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₅₋₆-cycloalkyl, and C₆₋₇-cycloalkyl.

The term “C₁₋₄-alkoxy” refers to a straight or branched C₁₋₄-alkyl group which is attached to the remainder of the molecule through an oxygen atom. For parts of the range C₁₋₄-alkoxy, all subgroups thereof are contemplated such as C₁₋₃-alkoxy, C₁₋₂-alkoxy, C₂₋₄-alkoxy, C₂₋₃-alkoxy and C₃₋₄-alkoxy. Examples of said C₁₋₄-alkoxy include methoxy, ethoxy, n-propoxy, isopropoxy, n-butoxy, isobutoxy, sec-butoxy and tert-butoxy.

The term “hydroxy-C₁₋₄-alkyl” denotes a straight or branched C₁₋₄-alkyl group that has one or more hydrogen atoms thereof replaced with OH. Examples of said hydroxy-C₁₋₄-alkyl include hydroxymethyl, 2-hydroxyethyl and 2,3-dihydroxypropyl.

The term “halo-C₁₋₄-alkyl” denotes a straight or branched C₁₋₄-alkyl group that has one or more hydrogen atoms thereof replaced with halogen. Examples of said halo-C₁₋₄-alkyl include fluoromethyl, trifluoromethyl, trichloromethyl and 2-fluoroethyl.

The term “cyano-C₁₋₄-alkyl” denotes a straight or branched C₁₋₄-alkyl group that has is one or more hydrogen atoms thereof replaced with cyano. Examples of said cyano-C₁₋₄-alkyl include cyanomethyl, 2-cyanoethyl and 3-cyanopropyl.

The term “amino-C₁₋₄-alkyl” denotes a straight or branched C₁₋₄-alkyl group substituted with an amino group. Examples of said amino-C₁₋₄-alkyl group include aminomethyl and 2-aminoethyl.

The term “C₁₋₄-alkylamino-C₁₋₄-alkyl” denotes an amino-C₁₋₄-alkyl group as defined above, wherein the amino group is substituted with a straight or branched C₁₋₄-alkyl group. Examples of said C₁₋₄-alkylamino-C₁₋₄-alkyl include methylaminoethyl and ethylaminopropyl.

The term “di(C₁₋₄-alkyl)amino-C₁₋₄-alkyl” denotes an amino-C₁₋₄-alkyl group as defined above, wherein the amino group is disubstituted with straight or branched C₁₋₄-alkyl groups, which can be the same or different. Examples of said di(C₁₋₄-alkyl)amino-C₁₋₄-alkyl include N,N-dimethylaminomethyl, N-ethyl-N-methylaminoethyl and N,N-diethylaminomethyl.

The terms “heteroaryl” and “heteroaromatic ring” denote a monocyclic heteroaromatic ring comprising 5 to 6 ring atoms in which one or more of the ring atoms are other than carbon, such as nitrogen, sulphur or oxygen. Examples of heteroaryl groups include furyl, pyrrolyl, thienyl, oxazolyl, isoxazolyl, imidazolyl, thiazolyl, isothiazolyl, pyridinyl, pyrimidinyl, tetrazolyl, pyrazolyl, pyridazinyl, pyrazinyl and thiadiazolyl.

The terms “heterocyclyl” and “heterocyclic ring” denote a non-aromatic, fully saturated or partially unsaturated, preferably fully saturated, monocyclic ring system having from 3 to 7 ring atoms, especially 5 or 6 ring atoms, in which one or more of the ring atoms are other than carbon, such as nitrogen, sulphur or oxygen. Examples of heterocyclic groups include piperidinyl, morpholinyl, homomorpholinyl, azepanyl, piperazinyl, oxo-piperazinyl, diazepinyl, tertahydropyridinyl, tetrahydropyranyl, pyrrolidinyl, tertrahydrofuranyl, and dihydropyrrolyl, groups.

The term “heterocyclic-C₁₋₄-alkyl” refers to a heterocyclic ring that is directly linked to a straight or branched C₁₋₄-alkyl group via a carbon or nitrogen atom of said ring. Examples of said heterocyclic-C₁₋₄-alkyl include piperidin-4-ylmethyl, piperidin-1-ylmethyl, morpholin-4-yl-methyl and piperazin-4-ylmethyl. The C₁₋₄-alkyl part, which includes methylene, ethylene, propylene or butylene, is optionally substituted by one or more substituents selected from halogen, amino, methoxy, or hydroxyl.

The term “C₁₋₃-alkylene” denotes a straight or branched divalent saturated hydrocarbon chain having from 1 to 3 carbon atoms. The C₁₋₃-alkylene chain may be attached to the rest of the molecule and to the radical group through one carbon within the chain or through any two carbons within the chain. Examples of C₁₋₃-alkylene radicals include methylene [—CH₂—], 1,2-ethylene [—CH₂—CH₂—], 1,1-ethylene [—CH(CH₃)—], 1,2-propylene [—CH₂—CH(CH₃)—] and 1,3-propylene [—CH₂—CH₂—CH₂—]. When referring to a “C₁₋₃-alkylene” radical, all subgroups thereof are contemplated, such as C₁₋₂-alkylene and C₂₋₃-alkylene.

“Halogen” refers to fluorine, chlorine, bromine or iodine, preferably fluorine and chlorine, most preferably fluorine.

“Hydroxy” refers to the —OH radical.

“Cyano” refers to the —CN radical.

“Oxo” refers to the carbonyl group ═O.

“Optional” or “optionally” means that the subsequently described event or circumstance may but need not occur, and that the description includes instances where the event or circumstance occurs and instances in which it does not.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

“Prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2^{nd} Ed., Elsevier Academic Press (2004), pp. 498-549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

Throughout the specification and the appended claims, a given chemical formula or name shall also encompass all salts, hydrates, solvates, N-oxides and prodrug forms thereof. Further, a given chemical formula or name shall encompass all tautomeric and stereoisomeric forms thereof. Tautomers include enol and keto forms. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in their pure cis or trans forms or as mixtures of those.

The compounds of formula (I) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### The Group X

In the compounds of the invention, X may be selected from any one of the radicals of formula 1-16.

Currently preferred embodiments of the invention include those where X is:

the formula 1 and R¹, R², Y, Z and W are as defined above; or
the formula 2 and R¹, R², Y, Z and W are as defined above; or
the formula 3 and R¹, R², Y, and Z are as defined above; or
the formula 4 and R¹, R², Y and W are as defined above; or
the formula 5 and R¹, R², and Y are as defined above; or
the formula 6 and R¹, R², Y, and Z are as defined above.

### The Group B

In an embodiment of the invention, B is a bond, O, NR⁴ such as NH, NCH₃, or NCH₂CH₃, —C(O)— or C₁₋₃alkylene such as methylene, ethylene or propylene radicals. In a currently preferred embodiment B is a bond, —C(O)— or methylene. In another preferred embodiment B is a bond.

### The Group Y

In a currently preferred embodiment of the invention Y is selected from hydrogen, hydroxyl, amino (NH₂), —NHR⁶ such as NHCH₃, NHCH₂CH₃, or —OCH₃. In another currently preferred embodiment Y is H, OH, or NH₂. In an alternative currently preferred embodiment Y is hydrogen

### The Group Z

Z is selected from hydrogen, fluorine, hydroxyl, C₁₋₄-alkoxy such as methoxy or ethoxy, halo-C₁₋₄-alkyl such as fluoromethoxy, difluoromethyoxy or trimethoxy, CONH₂, cyano, SO₂NH₂, amino, —NHR⁶ such as NHCH₃, NHCH₂CH₃. In a presently preferred embodiment of the invention Z is hydrogen or hydroxyl.

### The Group W

In a currently preferred embodiment of the invention W is selected from H, C₁₋₄-alkyl such as methyl, ethyl, propyl, isopropyl, or halo-C₁₋₄-alkyl such as fluoromethyl, difluoromethyl or trifluoromethyl. In another currently preferred embodiment W is hydrogen.

### The Group R¹

In one embodiment of the invention R¹ is phenyl or 6-membered heteroaryl such as pyridine, pyridazine, pyrimidine, pyrazine, optionally substituted with one or more substituents selected from halogen such as chloro or fluoro, cyano, C₁₋₄-alkyl such as methyl, ethyl, propyl or isopropyl, halo-C₁₋₄-alkyl such as fluoromethyl, difluoromethyl or trifluoromethyl, C₁₋₄alkoxy-C₁₋₄alkyl, hydroxy-C₁₋₄-alkyl such as hydroxylmethyl or hydroxylethyl, cyano-C₁₋₄-alkyl such as cyanomethyl or cyanoethyl, amino-C₁₋₄-alkyl such as aminomethyl, aminoethyl or aminopropyl, C₁₋₄-alkylamino-C₁₋₄-alkyl, di(C₁₋₄-alkyl)amino-C₁₋₄-alkyl, —NR^{4A}R⁴⁶, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵.

In a currently preferred embodiment of the invention R¹ is optional substituted with one or more substituents selected from halogen such as fluoro or chloro, cyano, hydroxyl, C₁₋₄-alkyl such as methyl or ethyl, halo-C₁₋₄-alkyl such as fluoromethyl, difluoromethyl or trifluoromethyl, C₁₋₄alkoxy-C₁₋₄alkyl, hydroxy-C₁₋₄-alkyl, cyano-C₁₋₄-alkyl such as cyanomethyl or cyanoethyl, amino-C₁₋₄-alkyl, C₁₋₄-alkylamino-C₁₋₄-alkyl, di(C₁₋₄-alkyl)amino-C₁₋₄-alkyl, —N^{4A}R^{4B}.

In another currently preferred embodiment R¹ is heteroaryl such as pyridine-2-yl, pyridine-3-yl or pyridine-4-yl optionally substituted with one or more substituents selected from as fluoro, chloro, and C₁₋₄-alkyl such as methyl, ethyl, propyl, or isopropyl.

In an alternative embodiment R¹ is phenyl, optionally substituted at one or more of the para-, meta- and ortho-positions by one or more substituents selected from hydrogen, fluoro, chloro, cyano, hydroxyl, C₁₋₄-alkyl such as methyl, ethyl, propyl or isopropyl, or fluoromethyl, difluoromethyl, or trifluoromethyl.

In a currently preferred embodiment R¹ is phenyl substituted at the para position by a substituent selected from, fluoro, chloro, cyano, hydroxyl, C₁₋₄-alkyl such as methyl, ethyl, propyl or isopropyl, or fluoromethyl, difluoromethyl, or trifluoromethyl. In an alternative currently preferred embodiment the para substituent is selected from fluoro, chloro or methyl.

In another currently preferred embodiment R¹ is phenyl substituted at the meta-position by hydrogen.

In a further currently preferred embodiment R¹ is phenyl substituted at the ortho position by a substituent selected from hydrogen, fluoro, methyl, fluoromethyl, difluoromethyl, or trifluoromethyl. In another preferred embodiment R¹ is phenyl substituted at the ortho position by hydrogen, fluoro or methyl.

In a currently preferred embodiment of the invention R¹ is a mono, di, or tri substituted phenyl ring wherein the ortho, meta and/or para positions may be any combination of the substituents discussed above.

In a preferred embodiment the optional substituents of R¹ have a length of 4 atoms or fewer, preferably of 3 atoms or fewer, more preferably of 2 atoms or fewer.

### The Group R²

In one currently preferred embodiment of the invention R² is —B-Q-[R³]ₙ. n can be 1, 2, 3, or 4. In another currently preferred embodiment n is 1 or 2.

The ring Q is a saturated or partially unsaturated monocyclic 3-7 membered heterocyclic or C₃₋₇-cycloalkyl ring substituted with R³. In a currently preferred embodiment Q is a 7-membered saturated or partially unsaturated 7-membered heterocyclic ring such as a homomorpholine ring, or a bridged homomorpholine ring wherein the bridge is formed by an ethylene or propylene radical, or a 7-membered cycloalkyl ring such as cycloheptane.

In an alternative preferred embodiment Q is a 5- or 6-membered saturated or partially unsaturated 5 or 6 membered heterocyclic such as tetrahydrofuranyl, tetrahydropyranyl, piperidinyl, piperazinyl, morpholinyl, cyclohexyl, or any of the foregoing rings comprising a bridge formed by an ethylene or propylene radical, or a 5 or 6-membered cycloalkyl ring such cyclopentyl or cyclohexyl. In an embodiment Q is piperidinyl, piperazinyl, or morpholinyl.

In a currently preferred embodiment R² is —B-Q-[R³]ₙ, wherein R³ is selected from:

(i) 3-7 membered heterocyclyl- such as 2-, or 4-pyrrolidyl, 2-, 3-, or 4-piperidinyl, 2-, or 3-piperazinyl, or 2- or 3-morpholinyl; 3-7 membered heterocyclyl-C₁₋₄-alkyl-such as piperidin-4-ylmethyl, piperidin-1-ylmethyl, morpholin-4-yl-methyl, morpholin-2-yl-methyl, and morpholin-3-yl-methyl and piperazin-4-ylmethyl, piperazin-2-ylmethyl or piperazin-3-ylmethyl, or piperidin-4-ylethyl, piperidin-1-ylethyl, morpholin-4-yl-ethyl, morpholin-2-yl-ethyl, and morpholin-3-yl-ethyl and piperazin-4-ylethyl, piperazin-2-ylethyl or piperazin-3-ylethyl, or piperidin-4-ylpropyl, piperidin-1-ylpropyl, morpholin-4-yl-propyl, morpholin-2-yl-propyl, and morpholin-3-yl-propyl and piperazin-4-ylpropyl, piperazin-2-ylpropyl or piperazin-3-ylpropyl, or piperidin-4-ylbutyl, piperidin-1-ylbutyl, morpholin-4-yl-butyl, morpholin-2-yl-butyl, and morpholin-3-yl-butyl and piperazin-4-ylbutyl, piperazin-2-ylbutyl or piperazin-3-ylbutyl; (3-7 membered heterocyclyl-C₁₋₄-alkyl)-amino-C₁₋₄-alkyl- such as (piperidine-4-ylmethyl)aminomethyl, amino-C₁₋₄-alkoxy-C₁₋₄-alkyl-, (amino-C₁₋₄-alkyl)-amino-C₁₋₄-alkyl)-, —C₁₋₄-alkyl-NR⁶C(O)OR⁵, —C₁₋₄-alkyl-NR⁶C(O)NR^{4A}R^{4B}, —C₁₋₄-alkyl-C(O)NR^{4A}R^{4B}, (3-7 membered heterocyclyl-C₁₋₄-alkyl)-C(O)—, —C₁₋₄-alkyl-C(O)OR⁵, —OC(O)R⁵, or
(ii) —C(O)NR^{9A}R^{9B} wherein R^{9A} and R^{9B} together with the nitrogen to which they are attached form a 3-7 membered cyclic amino group substituted with one or more substituents selected from: C₁₋₄-alkyl, C₁₋₄alkoxy-C₁₋₄alkyl-, C₃₋₇-cycloalkyl. In a preferred embodiment the cyclic amino group is pyrrolidyl, piperidinyl, piperazinyl, or morpholinyl each of which is substituted on a ring carbon or nitrogen atom by one or more substituents selected from methyl, ethyl, propyl, iso-propyl, n-butyl, sec-butyl, tert-butyl, methoxyethyl, cyclopropyl or cyclobutyl. In a currently preferred embodiment the cyclic amino group is piperazinyl substituted on the 4-position by methyl, ethyl, propyl, iso-propyl, sec-butyl, or cyclopropyl, or
(iii) —C(O)NR⁶R^{10B} wherein R^{10B} is 3-7 membered heterocyclyl- such as defined above, or 3-7 membered heterocyclyl-C₁₋₄-alkyl- such as defined above, or —C₁₋₄-alkyl-NR⁶C(O)R⁵; or
R^{10B} is 5 or 6 membered heteroaryl-C₁₋₄-alkyl- such as tetrazolylmethyl, wherein the heteroaryl ring is optionally substituted with one or more substituents selected from C₁₋₄-alkyl or halo-C₁₋₄-alkyl, and wherein the C₁₋₄-alkyl part of the heteroaryl-C₁₋₄-alkyl- group is optionally substituted by one or more C₁₋₄-alkyl- groups, or the C₁₋₄-alkyl part is substituted with two C₁₋₄-alkyl groups which, together with the carbon atom to which they are attached, join together to form a spiro 3-6 membered cycloalkyl ring. In a preferred embodiment R^{10B} is tetrazolylmethyl-, wherein the tetrazole group is optionally substituted with one or more substituents selected from C₁₋₄-alkyl or halo-C₁₋₄-alkyl, and wherein the methyl of the tetrazolylmethyl is substituted with two C₁₋₄-alkyl groups which, together with the carbon atom to which they are attached, join together to form a spiro cyclopropyl, cyclobutyl or cyclopentyl group.

The groups R^{4A}, R^{4B} and R⁵ are each independently selected from hydrogen, C₁₋₄-alkyl- such as methyl, ethyl, propyl, iso-propyl, n-butyl, sec-butyl, tert-butyl, 3-7 membered heterocyclyl-C₁₋₄-alkyl- as defined previously, amino-C₁₋₄-alkyl- such as aminomethyl, amino ethyl, 3-7 membered heterocyclyl- as defined above, —C₁₋₄-alkyl-NR⁶C(O)OR⁵, or C₃₋₇-cycloalkyl such as cyclopropyl, cyclobutyl, cyclopentyl, cyclohexyl,

or R^{4A} and R^{4B} together with the nitrogen to which they are attached form a 3-7 membered cyclic amino group such as pyrrolidyl, piperidinyl, homopiperidinyl, piperazinyl, homopiperizinyl or morpholinyl, optionally substituted by one or more substituents selected from: C₁₋₄-alkyl such as methyl, ethyl, propyl, iso-propyl, n-butyl, butyl, sec-butyl, tert-butyl, —NR^{4A}R^{4B} such as —NH₂, —NHCH₃, NHCH₂CH₃, or NH(CH₃)₂.

In the currently preferred embodiments the 3-7 membered heterocyclyl (other than the ring Q), or the heterocyclyl part of the 3-7 membered heterocyclyl-C₁₋₄-alkyl-, (3-membered heterocyclyl-C₁₋₄-alkyl)-amino-C₁₋₄-alkyl-, or (3-7 membered heterocyclyl-C₁₋₄-alkyl)-C(O)— group is optionally substituted with one or more substituents selected from oxo, C₁₋₄-alkyl- such as methyl, ethyl, propyl, iso-propyl, n-butyl, sec-butyl, tert-butyl, —C(O)OR⁵, —C(O)R⁵, —C(O)NR^{4A}R^{4B}, —NR^{4A}R^{4B} such as —NH₂, —NHCH₃, NHCH₂CH₃, or N(CH₃)₂, —C₁₋₄-alkyl-C(O)NR^{4A}R^{4B}, or C₁₋₄alkoxy-C₁₋₄alkyl such as methoxyethyl.

In a presently preferred embodiment the R³ group includes a divalent radical —C₁₋₄-alkyl- directly attached to the Q ring, such that R³ may be, for example 3-7 membered heterocyclyl-C₁₋₄-alkyl-, (3-7 membered heterocyclyl-C₁₋₄-alkyl)-amino-C₁₋₄-alkyl-, amino-C₁₋₄-alkoxy-C₁₋₄-alkyl-, (amino-C₁₋₄-alkyl)-amino-C₁₋₄-alkyl-, —C₁₋₄-alkyl-NR⁶C(O)OR⁵, —C₁₋₄-alkyl-NR⁶C(O)NR^{4A}R^{4B}, or —C₁₋₄-alkyl-C(O)NR^{4A}R^{4B}. In a preferred embodiment that —C₁₋₄-alkyl- radical is optionally substituted with one or more groups independently selected from halogen, amino, methoxy, and hydroxyl. In an embodiment the —C₁₋₄-alkyl- radical is selected from methylene, ethylene, propylene or butylene, any of which is optionally substituted by one or more groups independently selected from halogen, amino, methoxy, and hydroxyl. For example, the R³ group includes CH₂—C(O)NR^{4A}R^{4B}, —(CH₂)₂—C(O)NR^{4A}R^{4B}, —(CH₂)₃—C(O)NR^{4A}R^{4B} or 3-7 membered heterocyclyl-CH₂—, 3-7 membered heterocyclyl-(CH₂)₂—, or 3-7 membered heterocyclyl-(CH₂)₃—.

R⁴ and R⁶ are each independently selected from hydrogen or C₁₋₄-alkyl such as methyl, ethyl, propyl, iso-propyl, n-butyl, sec-butyl, tert-butyl; and

In another embodiment, R² is —B—R³ and R³ is —NR⁶R^{11B}, wherein R^{11B} is 3-7 membered heterocyclyl-C₁₋₄-alkyl- as defined previously and R⁶ is as defined previously;

In an alternative currently preferred embodiment R² is —B-Q-[R³]ₙ, and R³ is: (i) —C(O)NR⁶R^{10B} where R⁶ is methyl or hydrogen and R^{10B} is a 3-7 membered heterocyclyl- such as piperidinyl including piperidine-4-yl and 1-methylpiperidine-4-yl or R^{10B} is a 3-7 membered heterocyclyl-C₁₋₄-alkyl- including morpholine-4-ylmethyl, morpholine-4-ylethyl, morpholine-4-ylpropyl, piperidine-4-ylmethyl-, piperidine-4-ylethyl-, piperidine-4-ylpropyl-, piperazine-1-ylmethyl, or piperazine-1-ylethyl wherein the nitrogen atom in the piperidine 1-position or the piperazine 4-position is substituted with a substituent selected from hydrogen, methyl, ethyl, isopropyl, methoxyethyl-.

In a currently preferred embodiment R³ is —C₁₋₄-alkyl-C(O)NR^{4A}R^{4B} where R^{4A} is hydrogen and R^{4B} is amino ethyl, or R^{4A} and R^{4B} together with the nitrogen to which they are attached form a pyrrolidyl or piperidinyl ring optionally substituted by one or more substituents selected from —NH₂, —NHCH₃, NHCH₂CH₃, or N(CH₃)₂.

In a preferred embodiment R² is:

[chemical structure]

wherein
T is a trivalent nitrogen atom or a methyne (i.e. CH);
R⁶ is hydrogen or C₁₋₄-alkyl such as methyl
R^{10B} is 3-7 membered heterocyclyl- group such as morpholine or piperidine, or 3-7 membered heterocyclyl-C₁₋₄-alkyl- such as morphdlinylmethyl, morpholinylethyl, morpholinylpropyl, piperidinylmethyl, piperidinylethyl, piperidinylpropyl, piperazinylmethyl, piperazinylethyl or piperazinylpropyl any of which heterocyclic rings is optionally substituted by one or more substituents selected from C₁₋₄-alkyl- and C₁₋₄alkoxy-C₁₋₄alkyl.

In another embodiment R² is:

[chemical structure]

wherein
T is a trivalent nitrogen atom or a methyne (i.e. CH);
P is a direct bond or a diradical selected from methylene, ethylene, or propylene;
R⁶ is hydrogen or C₁₋₄-alkyl;
R¹² is selected from hydrogen, C₁₋₄-alkyl such as methyl, ethyl, propyl, butyl, isopropyl, and C₁₋₄alkoxy-C₁₋₄alkyl such as methoxyethyl-.

In another embodiment R² is:

[chemical structure]

wherein
T is a trivalent nitrogen atom or a methyne (i.e. CH);
P is a diradical selected from methylene, ethylene, or propylene;
R⁶ is hydrogen or C₁₋₄-alkyl;
R¹² is selected from hydrogen, C₁₋₄-alkyl such as methyl, ethyl, propyl, butyl, isopropyl, and C₁₋₄alkoxy-C₁₋₄alkyl such as methoxyethyl-.

In another embodiment R² is:

[chemical structure]

wherein
R³ is —C₁₋₄-alkyl-C(O)NR^{4A}R^{4B} such as —CH₂—C(O)NR^{4A}R^{4B}, (CH₂)₂—C(O)NR^{4A}R^{4B}, or —(CH₂)₃—C(O)NR^{4A}R^{4B} wherein R^{4A} and R^{4B} are each independently selected from hydrogen, C₁₋₄-alkyl- such as methyl, ethyl, propyl, and amino-C₁₋₄-alkyl-, or R^{4A} and R^{4B} together with the nitrogen to which they are attached form a 3-7 membered cyclic amino group such as pyrrolidine, piperidine, piperazine or morpholine, any of which is optionally substituted by one or more substituents selected from: C₁₋₄-alkyl, or —NR^{4A}R^{4B}.

In any of the compounds of the invention, the R¹ group may be any one of the specific R¹ groups of the corresponding position of any of the examples described herein.

In any of the compounds of the invention, the R² group may be any one of the specific R² groups of the corresponding position of any of the examples described herein.

In any of the compounds of the invention, the R³ group may be any one of the specific R³ groups of the corresponding position of any of the examples described herein.

Specific currently preferred embodiments of the invention include:
4-[1-(4-Chlorophenyl)-1H-pyrrolo[2,3-c]pyridin-3-yl]-N-(piperidin-4-ylmethyl)piperidine-1-carboxamide
4-[1-(4-Chlorophenyl)-1H-pyrrolo[2,3-c]pyridin-3-yl]-N-(1-methylpiperidin-4-yl)piperidine-1-carboxamide
4-[1-(4-Chlorophenyl)-1H-pyrrolo[2,3-c]pyridin-3-yl]-N-[(1-methylpiperidin-4-yl)methyl]piperidine-1-carboxamide
4-[1-(4-Chlorophenyl)-1H-pyrrolo[2,3-c]pyridin-3-yl]-N-[(1-ethylpiperidin-4-yl)methyl]piperidine-1-carboxamide
4-[1-(4-Chlorophenyl)-1H-pyrrolo[2,3-c]pyridin-3-yl]-N-methyl-N-[(1-methylpiperidin-4-yl)methyl]piperidine-1-carboxamide
4-[1-(4-Chlorophenyl)-1H-pyrrolo[2,3-c]pyridin-3-yl]-N-[2-(piperazin-1-yl)ethyl]piperidine-1-carboxamide
4-[1-(4-Chlorophenyl)-1H-pyrrolo[2,3-c]pyridin-3-yl]-N-[2-(1-methylpiperidin-4-yl)ethyl]piperidine-1-carboxamide
4-[1-(4-Chlorophenyl)-1H-pyrrolo[2,3-c]pyridin-3-yl]-N-[3-(morpholin-4-yl)propyl]piperidine-1-carboxamide
4-[1-(4-Chlorophenyl)-1H-pyrrolo[2,3-c]pyridin-3-yl]-N-{[1-(propan-2-yl)piperidin-4-yl]methyl}piperidine-1-carboxamide
4-[1-(4-Chlorophenyl)-1H-pyrrolo[2,3-c]pyridin-3-yl]-N-{[1-(2-methoxyethyl)piperidin-4-yl]methyl}piperidine-1-carboxamide
4-[1-(4-Chlorophenyl)-1H-pyrazolo[3,4-c]pyridin-3-yl]-N-[(1-methylpiperidin-4-yl)methyl]piperazine-1-carboxamide
N-(2-Aminoethyl)-2-{4-[1-(4-chlorophenyl)-1H-pyrazolo[3,4-c]pyridin-3-yl]morpholin-3-yl}acetamide
2-{4-[1-(4-Chlorophenyl)-1H-pyrazolo[3,4-c]pyridin-3-yl]morpholin-3-yl}-1-[(3S)-3-(dimethylamino)pyrrolidin-1-yl]ethan-1-one
or a pharmaceutically acceptable salt, or N-oxide thereof.

In one aspect, the invention relates to a compound of formula (I) for use in therapy. The compounds as defined above are useful as inhibitors of SSAO activity. As such, they are useful in the treatment or prevention of conditions and diseases in which inhibition of SSAO activity is beneficial. More specifically, they are useful for the treatment or prevention of inflammation, inflammatory diseases, immune or autoimmune disorders, cystic fibrosis, or inhibition of tumour growth.

In particular, it is believed that compounds of formula (I) are useful for the treatment or prevention of arthritis (such as rheumatoid arthritis, juvenile rheumatoid arthritis, osteoarthritis and psoriatic arthritis), synovitis, vasculitis, conditions associated with inflammation of the bowel (such as Crohn's disease, ulcerative colitis, inflammatory bowel disease and irritable bowel syndrome), atherosclerosis, multiple sclerosis, Alzheimer's disease, vascular dementia, pulmonary inflammatory diseases (such as asthma, chronic obstructive pulmonary disease and acute respiratory distress syndrome), fibrotic diseases (including idiopathic pulmonary fibrosis, cardiac fibrosis and systemic sclerosis (scleroderma)), inflammatory diseases of the skin (such as contact dermatitis, atopic dermatitis and psoriasis), systemic inflammatory response syndrome, sepsis, inflammatory and/or autoimmune conditions of the liver (such as autoimmune hepatitis, primary biliary cirrhosis, alcoholic liver disease, sclerosing cholangitis, and autoimmune cholangitis), diabetes (type I or II) and/or the complications thereof, chronic heart failure, congestive heart failure, ischemic diseases (such as stroke and ischemia-reperfusion injury), and myocardial infarction and/or the complications thereof.

It is believed that the compounds of the invention are especially useful for the treatment or prevention of vasculitis, including, but not limited to, giant cell arteritis, Takayasu's arteritis, Polyarteritis nodosa, Kawasaki disease, Wegener's granulomatosis, Churg-Strauss syndrome, microscopic polyangiitis, Henoch-Schnlein purpura, cryoglobulinemia, cutaneous leukocytoclastic angiitis and primary angiitis of the central nervous system.

It is also believed that the compounds of the invention are especially useful for the treatment of rheumatoid arthritis, chronic obstructive pulmonary disease or atopic dermatitis.

In view of the evidence cited in the above introduction that VAP-1 is up regulated in several cancers, including gastric cancer, melanoma, hepatoma and head and neck tumours and that mice bearing enzymatically inactive VAP-1 grow melanomas more slowly, and in view of the link between VAP-1 and angiogenesis, it is also expected that the compounds of the invention are anti-angiogenic and therefore have utility in the treatment of cancers by inhibition of tumour growth.

The invention thus includes the compounds of formula (I) above for use in the treatment or prevention of the above-mentioned conditions and diseases. The invention also includes the use of said compounds in the manufacture of a medicament for the treatment or prevention of the above-mentioned conditions and diseases. The invention furthermore includes methods for treatment or prevention of such conditions and diseases, comprising administering to a mammal, including man, in need of such treatment an effective amount of a compound as defined above.

Methods delineated herein include those wherein the subject is identified as in need of a particular stated treatment. Identifying a subject in need of such treatment can be in the judgment of a subject or a healthcare professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment is progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabeling/assay techniques, blotting/chemiluminescence methods, real-time PCR, and the like.

### Compositions

A currently preferred embodiment of the invention is a pharmaceutical composition comprising a compound of formula (I), together with one or more pharmaceutically acceptable carriers and/or excipients.

For clinical use, the compounds of the invention are formulated into pharmaceutical formulations for various modes of administration. It will be appreciated that compounds of the invention may be administered together with a physiologically acceptable carrier, excipient, or diluent. The pharmaceutical compositions of the invention may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the invention may be incorporated into is slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### Preparation of Compounds of the Invention

The compounds of formula (I) above may be prepared by, or in analogy with, conventional methods. The preparation of intermediates and compounds according to the Examples of the present invention may in particular be illuminated by the following Scheme's. Definitions of variables in the structures in Schemes herein are commensurate with those of corresponding positions in the formulas delineated herein.

[chemical structure]

wherein W, Y, Z, Q, R¹ and R³ are as defined in formula (I);

Compounds of general formula (Ia) can easily be prepared from 1H-pyrrolo[2,3-c]pyridines (IIa) by either introduction of the Q ring (or protected Q ring) followed by introduction of R¹ or by reversing these steps to give intermediates of general formula (IIIa). Compounds of general formula (IIIa) can then be converted to compounds of general formula (Ia) by standard synthetic methods. For example, condensation of 1H-pyrrolo[2,3-c]pyridines (IIa) with tert-butyl 4-oxopiperidine-1-carboxylate, reduction, introduction of R¹ by an arylation reaction and Boc de-protection can be used to give compounds of general formula (IVa). Functionalisation of compounds of general formula (IVa) by for example, urea formation, amide coupling or reductive amination gives compounds of general is formula (Ia).

[chemical structure]

wherein W, Y, Z, R¹ and R² are as defined in formula (I);

Compounds of general formula (Ib) can easily be prepared from bromoindoles (IIb) by either introduction of R² (for example by nucleophilic substitution) followed by R¹ (for example by a Suzuki reaction), or by reversing these steps (with an appropriate protecting group strategy).

[chemical structure]

wherein Y, Z, R¹ and R² are as defined in formula (I);

Compounds of general formula (Ic) can easily be prepared by reductive amination of 3-amino-pyridine-4-carbaldehydes of general formula (IIc) to give compounds of general formula (IIIc) and subsequent cyclisation to give pyrazolo[3,4-c]pyridines of general formula (IVc). Alternatively, pyrazolo[3,4-c]pyridines of general formula (IVc) can be prepared by cyclisation of (3-fluoropyridin-4-yl)carbonyl compounds of general formula (Vc) with hydrazine. Compounds of general formula (Ic) can be prepared from compounds of general formula (IVc) by standard N-arylation reactions.

[chemical structure]

wherein W, Y, R¹ and R² are as defined in formula (I);

Compounds of general formula (Id) can easily be prepared according to standard methods known in the scientific literature, for example, by lithiation of 7-bromo-4-chloro-5H-pyrrolo[3,2-d]pyrimidines (IId) and reaction with an aldehyde, followed by reduction and subsequent introduction of R¹ (for example by an arylation reaction). Such methods are known to those skilled in the art, for example in WO2008070507 and Antilla et al., JOC, 69, 5578, 2004.

[chemical structure]

wherein Y, R¹ and R² are as defined in formula (I);

Compounds of general formula (Ie) can easily be prepared according to standard methods known in the scientific literature, for example, by condensation of 5-chloropyrimidines (IIe) with a Weinreb amide and subsequent reaction with a hydrazine. Such methods are known to those skilled in the art, for example in WO2003039469 and Verma et al, Tet. Lett., 50, 383, 2009.

[chemical structure]

wherein Y, Z, R¹ and R² are as defined in formula (I);

Compounds of general formula (If) can easily be prepared by the condensation of pyrazine-2-carbonitriles with a Grignard reagent to give amine intermediates (IIf). Functionalisation of amines (IIf) to give amides or ureas of general formula (IIIf) and cyclisation with phosphorus oxychloride gives compounds of general formula (If).

[chemical structure]

wherein W, Z, R¹ and R² are as defined in formula (I);

Compounds of general formula (Ig) can be prepared from 1H-pyrrolo[2,3-d]pyridazines (IIg) by bromination with CuBr (for example, as described in Gallou et al., Syn. Lett., 2, 211-214, 2007) and subsequent introduction of R¹ (for example by an arylation reaction) and R² (for example by a Buchwald-Hartwig reaction).

[chemical structure]

wherein Y, Z, R¹ and R² are as defined in formula (I);

Compounds of general formula (Ih) can be prepared by sequential alkylations of 1H,2H,3H-imidazo[4,5-c]pyridin-2-ones (IIh), for example as described in WO2008054749.

[chemical structure]

wherein W, Y, Z, R¹ and R² are as defined in formula (I);

Compounds of general formula (Ii) can be prepared from 1H-pyrrolo[2,3-d]pyridazines (IIi) by bromination with CuBr (for example, as described in Gallou et al., Syn. Lett., 2, 211-214, 2007) followed by either introduction of R² (for example by nucleophilic substitution) followed by R¹ (for example by a Suzuki reaction), or by reversing these steps (with an appropriate protecting group strategy).

[chemical structure]

wherein W, Z, R¹ and R² are as defined in formula (I);

Compounds of general formula (Ij) can be prepared from 1H-pyrrolo[2,3-d]pyridazines (IIj) by bromination with CuBr (for example, as described in Gallou et al., Syn. Lett., 2, 211-214, 2007) followed by either introduction of R² (for example by nucleophilic substitution) followed by R¹ (for example by a Suzuki reaction), or by reversing these steps (with an appropriate protecting group strategy).

[chemical structure]

wherein Y, Z, R¹ and R² are as defined in formula (I);

Compounds of general formula (Ik) can be prepared by cyclisation of compounds of general formula (IIk) with hydrazines, for example, as described in Deeb et al., Journal of the Chinese Chemical Society, 37(3), 287-94; 1990.

[chemical structure]

wherein Z, R¹ and R² are as defined in formula (I);

Compounds of general formula (II) can be prepared by cyclisation of compounds of general formula (III) with hydrazines, for example, as described in Haider et al., Journal of the Chemical Society, Perkin Transactions 1: Organic and Bio-Organic Chemistry, 1, 169-72; 1986.

[chemical structure]

wherein W, Y, R¹ and R² are as defined in formula (I);

Compounds of general formula (Im) can be prepared by sequential alkylation arylation of 7H-pyrrolo[2,3-d]pyrimidines (IIm), for example as described in WO2009080682.

[chemical structure]

wherein Y, Z, R¹ and R² are as defined in formula (I);

Compounds of general formula (In) can be prepared by cyclisation of compounds of general formula (IIn) with hydrazines, for example, as described in Filaok et al., Journal of Organic Chemistry, 73(10), 3900-3906, 2008.

[chemical structure]

is wherein Z, R¹ and R² are as defined in formula (I);

Compounds of general formula (Io) can be prepared according to Scheme 15, for example as described in Haider et al., Journal of the Chemical Society, Perkin Transactions 1: Organic and Bio-Organic Chemistry, 1, 169-72; 1986.

[chemical structure]

wherein R¹ and R² are as defined in formula (I);

Compounds of general formula (Ip) can be prepared according to Scheme 16, for example as described in WO2007134828.

Optionally, a compound of formula (I) can also be transformed into another compound of formula (I) in one or more synthetic steps.

The following abbreviations have been used:

Ac acetyl
Ac₂O acetic anhydride
AcOH acetic acid
aq aqueous
Ar aryl
Boc tert-butoxycarbonyl
nBuLi n-butyllithium
calcd calculated
CDI carbonyldiimidazole
conc concentrated
d day
DCE dichloroethane
DCM dichloromethane
DIBALH diisobutylaluminium hydride
DIPEA diisopropylethylamine
DMAP 4-dimethylaminopyridine
DMF dimethylformamide
EDC 1-ethyl-3-(3-dimethylaminopropyl)carbodiimide hydrochloride
ES+ electrospray ionization
EtOAc ethyl acetate
EtOH ethanol

### Ex Example

h hour(s)
HBTU O-benzotriazole-N,N,N′,N′-tetramethyl-uronium-hexafluoro-phosphate
HOBt 1-hydroxybenzotriazole hydrate

### HPLC High Performance Liquid Chromatography

### HRMS High-Resolution Mass Spectrometry

### Int Intermediate

### LCMS Liquid Chromatography Mass Spectrometry

LDA lithium diisopropylamide
M molar
Me methyl
mCPBA meta-chloroperbenzoic acid
MeCN acetonitrile
MeOH methanol
min minute(s)
Ms methanesulfonate

### MS Mass Spectrometry

NaBH(OAc)₃ sodium triacetoxyborohydride

### NIS N-iodosuccinimide

### NMP N-methylpyrrolidone

Rf Retention time
RT room temperature
sat saturated

### SCX Strong Cation Exchange

SM starting material
TFA trifluoroacetic acid
THF tetrahydrofuran

### EXAMPLES AND INTERMEDIATE COMPOUNDS

### Experimental Methods

Reactions were conducted at room temperature unless otherwise specified. Microwave reactions were performed with a Biotage microwave reactor using process vials fitted with aluminum caps and septa. Hydrogenations were performed using a Thales H-Cube. Preparative flash chromatography was performed on Merck silica gel 60 (230-400 mesh) or using a Flash Master Personal system equipped with Strata SI-1 silica gigatubes, or using a CombiFlash Companion system equipped with RediSep silica columns. Reverse phase column chromatography was performed on a Gilson system (Gilson 321 pump and Gilson FC204 fraction collector) equipped with Merck LiChroprep® RP-18 (40-63 um) columns. Reverse Phase HPLC was performed on a Gilson system with a UV detector equipped with Phenomenex Synergi Hydro RP 150×10 mm, or YMC ODS-A 100/150×20 mm columns. The purest fractions were collected, concentrated and dried under vacuum. Compounds were typically dried in a vacuum oven at 40° C. prior to purity analysis. Compound analysis was performed by HPLC/LCMS using an Agilent 1100 HPLC system/Waters ZQ mass spectrometer connected to an Agilent 1100 HPLC system with a Phenomenex Synergi, RP-Hydro column (150×4.6 mm, 4 μm, 1.5 mL per min, 30° C., gradient 5-100% MeCN (+0.085% TFA) in water (+0.1% TFA) over 7 min, 200-300 nm). Accurate masses (HRMS) were measured using a Thermo Scientific LTQ Orbitrap XL equipped with an Advio TriVersa NanoMate electrospray ion source (during the analyses the calibration was checked by three masses. Spectra were acquired in positive electrospray mode. The acquired mass range was m/z 100-2000. Samples were dissolved in DMSO to give 10 mM solutions which were then further diluted with MeOH or 10 mM NH₄OAc in MeOH to −0.1 M solutions prior to analysis). The values reported correspond to the protonated molecular ions [MH]⁺. The compounds prepared were named using ACD Name 6.0, 7.0 or 10.0.

### Intermediate 1

### tert-Butyl 4-{1H-pyrrolo[2,3-c]pyridin-3-yl}-1,2,3,6-tetrahydropyridine-1-carboxylate

[chemical structure]

6-Azaindole (4.48 g, 37.9 mmol) was dissolved in MeOH (70 mL) and KOH (4.68 g, 83.4 mmol) and tert-butyl 4-oxopiperidine-1-carboxylate (8.31 g, 41.7 mmol) were added. The reaction mixture was heated at 70° C. for 18 h. The residue was partitioned between water (250 mL) and DCM (250 mL) and the aq phase was extracted with DCM (2×250 mL). The combined organic fractions were dried (MgSO₄) and concentrated in vacuo to give the title compound as a yellow foam (11.3 g, 99%). LCMS (ES+): 300.1 [MH]⁺.

### Intermediate 2

### tert-Butyl 4-{1H-pyrrolo[2,3-c]pyridin-3-yl}piperidine-1-carboxylate

[chemical structure]

Intermediate 1 (11.3 g, 37.7 mmol) was dissolved in EtOH (200 mL) and hydrogenated over 10% PdC in an H-cube at 90° C. and 90 bar. The reaction mixture was concentrated in vacuo to give the title compound as a yellow solid (11.1 g, 97%). LCMS (ES⁺): 302.1 [MH]⁺.

### Intermediate 3

### tert-Butyl 4-[1-(4-chlorophenyl)-1H-pyrrolo[2,3-c]pyridin-3-yl]piperidine-1-carboxylate

[chemical structure]

Intermediate 2 (11.1 g, 36.7 mmol) was dissolved in DMF (60 mL) and 1-chloro-4-iodo-benzene (10.5 g, 44.0 mmol), N,N′-dimethylethylenediamine (789 μL, 7.33 mmol), K₃PO₄ (16.3 g, 77.0 mmol) and CuI (698 mg, 3.67 mmol) were added under nitrogen. The reaction mixture was heated in a microwave at 160° C. for 20 min and concentrated in vacuo. The residue was partitioned between water (250 mL) and DCM (250 mL) and the aq phase was extracted with DCM (2×250 mL). The combined organic fractions were dried (MgSO₄) and concentrated in vacuo. The residue was purified by column chromatography to give the title compound as a yellow solid (6.86 g, 45%). LCMS (ES+): 411.9 [MH]⁺, HPLC: Rt 5.91 min, 76% purity.

### Intermediate 4

### 4-[1-(4-Chlorophenyl)-1H-pyrrolo[2,3-c]pyridin-3-yl]piperidine

[chemical structure]

Intermediate 3 (6.86 g, 16.6 mmol) was dissolved in DCM (200 mL) and TFA (50 mL) and stirred for 2 h. The solvents were removed in vacuo and the residue was dissolved in 1 M aq Na₂CO₃ (200 mL) and extracted with DCM (3×200 mL). The combined organic fractions were dried (MgSO₄) and concentrated in vacuo. The residue was purified by column chromatography to give the title compound as a red gum (3.18 g, 61%). LCMS (ES⁺): 312.1 [MH]⁺. HPLC: Rt 3.61 min, 96% purity.

### Intermediate 5

### 4-[1-(4-Chlorophenyl)-1H-pyrrolo[2,3-c]pyridin-3-yl]-N-(piperidin-4-ylmethyl)piperidine-1-carboxamide

[chemical structure]

CDI (936 mg, 5.77 mmol) was dissolved in DCM (50 mL), a solution of tert-butyl 4-(aminomethyl)piperidine-1-carboxylate (1.24 g, 5.77 mmol) and DIPEA (1.25 mL, 7.22 mmol) in DCM (10 mL) was added and the reaction mixture was stirred for 18 h. A solution of Intermediate 4 (1.50 g, 4.81 mmol) and DIPEA (1.25 mL, 7.22 mmol) in DCM (10 mL) was added and the reaction mixture was stirred for 24 h, diluted with 1 M aq Na₂CO₃ (100 mL) and extracted with DCM (3×100 mL). The combined organic fractions were dried (MgSO₄) and concentrated in vacuo. The residue was purified by column chromatography, dissolved in DCM (10 mL) and TFA (2.5 mL) and stirred for 1 h. The reaction mixture was concentrated in vacuo and the residue was dissolved in 1 M aq Na₂CO₃ (50 mL) and extracted with DCM (3×50 mL). The combined organic fractions were dried (MgSO₄) and concentrated in vacuo. The residue was purified by column chromatography to give the title compound as a pale yellow solid (1.41 g, 65%). LCMS (ES⁺): 452.0 [MH]⁺. HPLC: Rt 3.98 min, 97% purity.

### Intermediate 6

### N-(3-Aminopropyl)-4-[1-(4-chlorophenyl)-1H-pyrrolo[2,3-c]pyridin-3-yl]piperidine-1-carboxamide

[chemical structure]

Intermediate 6 (178 mg, 8%) was prepared similarly to Intermediate 5, using tert-butyl N-(3-aminopropyl)carbamate instead of tert-butyl 4-(aminomethyl)piperidine-1-carboxylate. LCMS (ES⁺): 412.3 [MH]⁺. HPLC: Rt 3.82 min, 100% purity.

### Intermediate 7

### tert-Butyl N-({4-[(3-aminopyridin-4-yl)methyl]morpholin-2-yl}methyl)carbamate

[chemical structure]

3-Amino-pyridine-4-carbaldehyde (513 mg, 4.20 mmol) was dissolved in DCE (7.3 mL) and tert-butyl(morpholin-2-ylmethyl)carbamate (999 mg, 4.62 mmol) and NaBH(OAc)₃ (1.07 g, 5.04 mmol) were added. The reaction mixture was heated using a microwave at 60° C. for 5 min, diluted with DCM (10 mL) and quenched with sat aq Na₂CO₃ (5 mL).

The aq phase was extracted with DCM (3×20 mL) and the combined organic fractions were dried (MgSO₄) and concentrated in vacuo to give the crude title compound as a yellow gum (1.37 g, 100%). LCMS (ES⁺): 323.1 [MH]⁺.

### Intermediate 8

### tert-Butyl N-[(4-{1H-pyrazolo[3,4-c]pyridin-3-yl}morpholin-2-yl)methyl]carbamate

[chemical structure]

Intermediate 7 (1.35 g, 4.20 mmol) was dissolved in AcOH (55 mL), and a solution of NaNO₂ (290 mg, 4.20 mmol) in water (438 μL) was added. The reaction mixture was stirred for 5 min and concentrated in vacuo. The residue was dissolved in EtOAc (40 mL) and washed with sat aq Na₂CO₃ (2×20 mL). The organic fraction was dried (MgSO₄) and concentrated in vacuo to give the crude title compound as a yellow gum (999 mg, 71%). LCMS (ES+): 334.0 [MH]⁺.

### Intermediate 9

### tert-Butyl N-({4-[1-(4-methylphenyl)-1H-pyrazolo[3,4-c]pyridin-3-yl]morpholin-2-yl}methyl)carbamate

[chemical structure]

Intermediate 8 (999 mg, 3.00 mmol) was dissolved in DMF (4 mL) and 1-methyl-4-iodobenzene (784 mg, 3.60 mmol), N,N′-dimethylethylenediamine (64.5 μL, 0.60 mmol), K₃PO₄ (1.34 g, 6.29 mmol) and CuI (57.1 mg, 0.30 mmol) were added. The reaction mixture was heated using a microwave at 140° C. for 20 min. The solvents were removed in vacuo and the residue was purified by column chromatography to give the crude title compound as a yellow gum (438 mg, 35%). LCMS (ES⁺): 424.0 [MH]⁺.

### Intermediate 10

### {4-[1-(4-Methylphenyl)-1H-pyrazolo[3,4-c]pyridin-3-yl]morpholin-2-yl}methanamine dihydrochloride

[chemical structure]

Intermediate 9 (438 mg, 1.03 mmol) was dissolved in 1.25 M HCl in EtOH (10 mL) and stirred overnight. The solvents were removed in vacuo to give the crude title compound as an orange gum (400 mg, 98%). LCMS (ES⁺): 324.0 [MH]⁺.

### Intermediate 11

### tert-Butyl 2-[(acetyloxy)methyl]morpholine-4-carboxylate

[chemical structure]

Ac₂O (5.17 mL, 54.7 mmol) was dissolved in DCM (200 mL), DMAP (611 mg, 5.00 mmol), DIPEA (9.52 mL, 54.7 mmol) and tert-butyl-2-(hydroxymethyl) morpholine-4-carboxylate (10.0 g, 49.7 mmol) were added and the reaction mixture was stirred for 1 h. The reaction mixture was washed with sat aq NH₄Cl (3×100 mL) and the aq fraction was extracted with DCM (2×100 mL). The combined organic fractions were dried (MgSO₄) and concentrated in vacuo to give the crude title compound as an off white solid (14.1 g). LCMS (ES⁺): 282.1 [MNa]⁺.

### Intermediate 12

### {4-[(3-Aminopyridin-4-yl)methyl]morpholin-2-yl}methyl acetate

[chemical structure]

Intermediate 11 (12.8 g, 49.5 mmol) was dissolved in TFA (20 mL) and DCM (80 mL) and the reaction mixture was stirred overnight and concentrated in vacuo. The residue was dissolved in DCE (80 mL), cooled to 0° C. and Et₃N (6.90 mL, 49.5 mmol) was added drop-wise. 3-Amino-pyridine-4-carbaldehyde (6.04 g, 49.5 mmol) and MeOH (50 mL) were added and the reaction mixture was stirred for 30 min. NaBH(OAc)₃ (12.6 g, 59.4 mmol) was added portion-wise and the reaction mixture was stirred overnight. Further 3-amino-pyridine-4-carbaldehyde (6.04 g, 49.5 mmol) and NaBH(OAc)₃ (25.2 g, 119 mmol) were added portion-wise over 2 d. The reaction mixture was stirred at 60° C. for 30 h, cooled to 0° C., quenched with sat aq Na₂CO₃ (50 mL) and diluted with DCM (100 mL). The organic fraction was washed with sat aq Na₂CO₃ (20 mL) and sat aq NH₄Cl (2×20 mL), dried (MgSO₄) and concentrated in vacuo. The residue was purified by filtration through a pad of silica to give the crude title compound as a yellow gum (5.77 g, 44%). LCMS (ES⁺): 266.1 [MH]⁺.

### Intermediate 13

### (4-{1H-Pyrazolo[3,4-c]pyridin-3-yl}morpholin-2-yl)methyl acetate

[chemical structure]

Intermediate 12 (5.77 g, 21.8 mmol) was dissolved in AcOH (282 mL), cooled to 0° C., and a solution of NaNO₂ (1.50 g, 21.8 mmol) in water (2.29 mL) was added. The reaction mixture was stirred for 5 min and concentrated in vacuo. The residue was dissolved in EtOAc (200 mL) and washed with sat aq Na₂CO₃ (2×100 mL), dried is (MgSO₄) and concentrated in vacuo to give the title compound as a dark yellow gum (3.85 g, 64%). LCMS (ES+): 277.1 [MH]⁺.

### Intermediate 14

### {4-[1-(4-Methylphenyl)-1H-pyrazolo[3,4-c]pyridin-3-yl]morpholin-2-yl}methyl acetate

[chemical structure]

Intermediate 13 (3.85 g, 13.9 mmol) was dissolved in DMF (20 mL) and 1-methyl-4-iodo-benzene (3.64 g, 16.7 mmol), N,N′-dimethylethylenediamine (300 μL, 2.78 mmol), K₃PO₄ (6.21 g, 29.2 mmol) and CuI (265 mg, 1.39 mmol) were added. The reaction mixture was heated in a microwave reactor at 140° C. for 1 h and concentrated in vacuo. The residue was purified by column chromatography to give the title compound as a yellow gum (260 mg, 5%). LCMS (ES⁺): 367.0 [MH]⁺. HPLC: Rt 5.07 min, 97.4%.

### Intermediate 15

### {4-[1-(4-Chlorophenyl)-1H-pyrazolo[3,4-c]pyridin-3-yl]morpholin-2-yl}methyl acetate

[chemical structure]

Intermediate 13 (200 mg, 0.72 mmol), 4-chlorophenylboronic acid (226 mg, 1.45 mmol), Cu(OAc)₂ (263 mg, 1.45 mmol) and pyridine (292 μL, 3.62 mmol) were suspended in DCM (10 mL) and stirred for 36 h. The reaction mixture was concentrated in vacuo and purified by column chromatography to give the crude title compound as a yellow solid (80.0 mg, 29%). LCMS (ES⁺): 387.0 [MH]⁺.

### Intermediate 16

### {4-[1-(4-Methylphenyl)-1H-pyrazolo[3,4-c]pyridin-3-yl]morpholin-2-yl}methanol

[chemical structure]

Intermediate 14 (200 mg, 0.55 mmol) was dissolved in MeOH (4 mL) and K₂CO₃ (302 mg, 2.18 mmol) was added. The reaction mixture was stirred for 30 min and concentrated in vacuo. The residue was dissolved in DCM (20 mL) and water (10 mL) and the aq phase was extracted with DCM (3×50 mL). The combined organic fractions were dried (MgSO₄) and concentrated in vacuo to yield the title compound as a dark brown gum (168 mg, 95%). LCMS (ES⁺): 325.1 [MH]⁺.

### Intermediate 17

### {4-[1-(4-Chlorophenyl)-1H-pyrazolo[3,4-c]pyridin-3-yl]morpholin-2-yl}methanol

[chemical structure]

Intermediate 17 (65.0 mg, 91%) was prepared similarly to Intermediate 16, using Intermediate 15 instead of Intermediate 14. LCMS (ES⁺): 345.0 [MH]⁺.

### Intermediate 18

### {4-[1-(4-Methylphenyl)-1H-pyrazolo[3,4-c]pyridin-3-yl]morpholin-2-yl}methyl methanesulfonate

[chemical structure]

Intermediate 16 (292 mg, 0.90 mmol) was dissolved in DCM (7 mL), cooled to 0° C. and Et₃N (138 μL, 0.99 mmol) and methanesulfonyl chloride (76.6 μL, 0.99 mmol) were added. The reaction mixture was stirred for 1 h, diluted with DCM (10 mL) and washed with sat aq NH₄Cl (2×5 mL) and sat aq Na₂CO₃ (2×5 mL). The organic fraction was dried (MgSO₄) and concentrated in vacuo to give the title compound as a brown gum (277 mg, 77%). LCMS (ES⁺): 403.0 [MH]÷.

### Intermediate 19

### {4-[1-(4-Chlorophenyl)-1H-pyrazolo[3,4-c]pyridin-3-yl]morpholin-2-yl}methyl methanesulfonate

[chemical structure]

Intermediate 19 (60.0 mg, 75%) was prepared similarly to Intermediate 18, using Intermediate 17 instead of Intermediate 16. LCMS (ES⁺): 422.9 [MH]⁺.

### Intermediate 20

### tert-Butyl 4-({4-[(3-aminopyridin-4-yl)methyl]morpholin-2-yl}methyl)piperazine-1-carboxylate

[chemical structure]

3-Amino-pyridine-4-carbaldehyde (584 mg, 4.78 mmol) was dissolved in DCM (10 mL) and tert-butyl 4-(morpholin-2-ylmethyl)piperazine-1-carboxylate (1.50 g, 5.26 mmol) and NaBH(OAc)₃ (1.11 g, 5.26 mmol) were added. The reaction mixture was heated in a microwave at 60° C. for 2.5 min, diluted with DCM (20 mL) and quenched with sat aq Na₂CO₃ (10 mL). The organic fraction was washed with sat aq NH₄Cl (10 mL). The combined aq fractions were extracted with DCM (2×20 mL) and the combined organic fractions were dried (MgSO₄) and concentrated in vacuo to give the crude title compound as a yellow gum (2.46 g). LCMS (ES⁺): 392.1 [MH]⁺.

### Intermediate 21

### tert-Butyl 4-[(4-{1H-pyrazolo[3,4-c]pyridin-3-yl}morpholin-2-yl)methyl]piperazine-1-carboxylate

[chemical structure]

Intermediate 20 (1.87 g, 4.76 mmol) was dissolved in AcOH (62 mL), cooled to 0° C. and a solution of NaNO₂ (330 mg, 4.76 mmol) in water (502 μL) was added. The reaction mixture was stirred for 5 min and concentrated in vacuo. The residue was dissolved in EtOAc (100 mL) and washed with sat aq Na₂CO₃ (2×50 mL). The organic fraction was dried (MgSO₄) and concentrated in vacuo to yield the title compound as a brown gum (1.80 g, 93%). LCMS (ES+): 403.1 [MH]⁺.

### Intermediate 22

### tert-Butyl 4-({4-[1-(4-chlorophenyl)-1H-pyrazolo[3,4-c]pyridin-3-yl]morpholin-2-yl}methyl)piperazine-1-carboxylate

[chemical structure]

Intermediate 21 (1.08 g, 2.69 mmol), 4-chlorobenzeneboronic acid (840 mg, 5.37 mmol), Cu(OAc)₂ (976 mg, 5.37 mmol) and pyridine (1.08 mL, 13.4 mmol) were suspended in DCE (19 mL) and stirred overnight. The solvents were removed in vacuo and the residue was purified by column chromatography to give the title compound as a pale yellow solid (58.0 mg, 4%). LCMS (ES⁺): 513.0 [MH]⁺. HPLC: Rt 4.81 min, 97.1% purity.

### Intermediates 23 to 30

Intermediates 23-30 were prepared similarly to Intermediate 20, by reductive amination of 3-amino-pyridine-4-carbaldehyde with the appropriate amine; see Table 1 below.

TABLE 1
Reductive aminations of 3-amino-pyridine-4-carbaldehyde

[chemical structure]

| | | Crude | LCMS | |
|---|---|---|---|---|
| Int | Structure | yield | (ES⁺) | Intermediate Name |
| 23 | [chemical structure] | 3.16 g 63% | 266.1 [MH]⁺ | Methyl 2-{4-[(3-aminopyridin-4- yl)methyl]morpholin-3-yl}acetate |
| 24 | [chemical structure] | 11.3 g 100% | 264.1 [MH]⁺ | Ethyl 1-[(3-aminopyridin-4- yl)methyl]piperidine-4-carboxylate |
| 25 | [chemical structure] | 541 mg 70% | 237.1 [MH]⁺ | 4-({[2-(Morpholin-4-yl)ethyl] amino}methyl)pyridin-3-amine |
| 26 | [chemical structure] | 585 mg 53% | 336.1 [MH]⁺ | tert-Butyl-4-(2-{[(3-aminopyridin-4- yl)methyl]amino}ethyl)piperazine- 1-carboxylate |
| 27 | [chemical structure] | 861 mg 42% | 250.1 [MH]⁺ | 1-[(3-Aminopyridin-4- yl)methyl]piperidin-4-yl acetate |
| 28 | [chemical structure] | 165 mg 36% | 293.3 [MH]⁺ | 4-{[3-(Morpholin-4- ylmethyl)morpholin-4-yl]methyl} pyridin-3-amine |
| 29 | [chemical structure] | 1.67 g 70% | 293.1 [MH]⁺ | tert-Butyl 4-[(3-aminopyridin-4- yl)methyl]piperazine-1-carboxylate |
| 30 | [chemical structure] | 1.76 g 40% | 250.1 [MH]⁺ | Methyl 1-[(3-aminopyridin-4- yl)methyl]piperidine-2-carboxylate |

### Intermediate 31

### tert-Butyl N-[(3-aminopyridin-4-yl)methyl]-N-[2-(morpholin-4-yl)ethyl]carbamate

[chemical structure]

Intermediate 25 (341 mg, 1.44 mmol) was, dissolved in DCM (15 mL), Boc₂O (346 mg, 1.59 mmol) was added and the reaction mixture was stirred for 1.5 h. The reaction mixture was quenched with sat aq Na₂CO₃ (40 mL) and the aq fraction was extracted with DCM (2×20 mL). The combined organic fractions were washed with brine (30 mL), dried (MgSO₄) and concentrated in vacuo to give the crude title compound as a brown oil (329 mg). LCMS (ES⁺): 337.0 [MH]⁺.

### Intermediate 32

### tert-Butyl 4-(2-{[(3-aminopyridin-4-yl)methyl][(tert-butoxy)carbonyl]amino}ethyl) piperazine-1-carboxylate

[chemical structure]

Intermediate 32 was prepared similarly to Intermediate 31, using Intermediate 26 instead of Intermediate 25, to give the title compound as a brown oil (409 mg, 54%). LCMS (ES⁺): 436.1 [MH]⁺. HPLC: Rt 4.18 min, 93% purity.

### Intermediates 33 to 40

Intermediates 33-30 were prepared similarly to Intermediate 21, by cyclisation of 3-aminopyridines 23-24 and 27-32 with NaNO₂; see Table 2 below.

TABLE 2
Cyclisation of 3-aminopyidines

[chemical structure]

| | | SM/ | | |
|---|---|---|---|---|
| | | Crude | LCMS | |
| Int | Structure | yield | (ES⁺) | Intermediate Name |
| 33 | [chemical structure] | Int 23 2.00 g 100% | 277.1 [MH]⁺ | 2-(4-{1H-Pyrazolo[3,4-c]pyridin-3- yl}morpholin-3-yl)acetate |
| 34 | [chemical structure] | Int 24 12.2 g 100% | 275.1 [MH]⁺ | Ethyl 1-{1H-pyrazolo[3,4-c]pyridin- 3-yl}piperidine-4-carboxylate |
| 35 | [chemical structure] | Int 31 243 mg 72% | 348.1 [MH]⁺ | tert-Butyl N-[2-(morpholin-4- yl)ethyl]-N-{1H-pyrazolo[3,4- c]pyridin-3-yl}carbamate |
| 36 | [chemical structure] | Int 32 399 mg 94% | 447.1 [MH]⁺ | tert-Butyl 4-(2-{[(tert- butoxy)carbonyl](1H-pyrazolo[3,4- c]pyridin-3-yl)amino}ethyl) piperazine-1-carboxylate |
| 37 | [chemical structure] | Int 27 618 mg 69% | 261.1 [MH]⁺ | 1-{1H-Pyrazolo[3,4-c]pyridin-3- yl}piperidin-4-yl acetate |
| 38 | [chemical structure] | Int 28 92.0 mg crude | 304.1 [MH]⁺ | 3-(Morpholin-4-ylmethyl)-4-{1H- pyrazolo[3,4-c]pyridin-3- yl}morpholine |
| 39 | [chemical structure] | Int 29 933 mg 54% | 304.2 [MH]⁺ | tert-Butyl 4-{1H-pyrazolo[3,4- c]pyridin-3-yl}piperazine-1- carboxylate |
| 40 | [chemical structure] | Int 30 650 mg 89% | 261.1 [MH]⁺ | Methyl 1-{1H-pyrazolo[3,4-c] pyridin-3-yl}piperidine-2- carboxylate |

### Intermediates 41 to 43

Intermediates 41-43 were prepared similarly to Intermediate 22, by N-arylation of 1H-pyrazolo[3,4-c]pyridines; see Table 3 below.

TABLE 3
N-Arylation of 1H-pyrazolo[3,4-c]pyridines

[chemical structure]

| | | Int/ | | |
|---|---|---|---|---|
| | | Crude | LCMS | |
| Int | Structure | yield | (ES⁺) | Intermediate Name |
| 41 | [chemical structure] | Int 34 619 mg 13% | 384.9 [MH]⁺ | Ethyl 1-[1-(4-chlorophenyl)-1H- pyrazolo[3,4-c]pyridin-3- yl]piperidine-4-carboxylate |
| 42 | [chemical structure] | Int 39 80.0 mg 75% | 414.0 [MH]⁺ | tert-Butyl 4-[1-(4-chlorophenyl)- 1H-pyrazolo[3,4-c]pyridin-3- yl]piperazine-1-carboxylate |
| 43 | [chemical structure] | Int 40 260 mg 28% | 370.9 [MH]⁺ | Methyl 1-[1-(4-chlorophenyl)-1H- pyrazolo[3,4-c]pyridin-3- yl]piperidine-2-carboxylate |

### Intermediate 44

### 1-[1-(4-Chlorophenyl)-1H-pyrazolo[3,4-c]pyridin-3-yl]piperidine-4-carboxylic acid hydrochloride

[chemical structure]

Intermediate 41 (834 mg, 2.17 mmol) was dissolved in 1:1 THF/water (16 mL), LiOH.H₂O (200 mg, 4.77 mmol) was added and the reaction mixture was stirred for 3 h. The THF was removed in vacuo and the reaction mixture was acidified to pH 1 with 1 M aq HCl (5 mL). The precipitate was collected by filtration and washed with water to give the title compound as an orange solid (450 mg, 53%). LCMS (ES⁺): 357.0 [MH]⁺. HPLC: Rt 4.92 min, 99.6% purity.

### Intermediate 45

### 4-({1-[1-(4-Chlorophenyl)-1H-pyrazolo[3,4-c]pyridin-3-yl]piperidin-4-yl}carbonyl)morpholine

[chemical structure]

Intermediate 44 (200 mg, 0.51 mmol) was dissolved in DMF (2 mL), HBTU (231 mg, 0.61 mmol) was added and the reaction mixture was stirred for 30 min. Morpholine (53.4 μL, 0.61 mmol) and DIPEA (266 μL, 1.53 mmol) were added and the reaction mixture was stirred overnight. The solvents were removed in vacuo and the residue was diluted with EtOAc (25 mL), washed with sat aq NH₄Cl (4×25 mL), dried (MgSO₄) and concentrated in vacuo. The residue was purified by column chromatography to give the title compound as a light yellow solid (78.7 mg, 36%). HRMS (ESI+) calcd for C22H24ClN5O2 426.1691, found 426.1691. HPLC: Rt 4.96 min, 100% purity.

### Intermediate 46

### tert-Butyl 4-({1-[1-(4-chlorophenyl)-1H-pyrazolo[3,4-c]pyridin-3-yl]piperidin-4-yl}carbonyl)piperazine-1-carboxylate

[chemical structure]

Intermediate 46 was prepared similarly to Intermediate 45, using tert-butyl 1-piperazinecarboxylate instead of morpholine to give the title compound as yellow gum (260 mg, 97%). LCMS (ES+): 525.1 [MH]⁺. HPLC: Rt 6.14 min, 100% purity.

### Intermediate 47

### 1-[1-(4-Chlorophenyl)-1H-pyrazolo[3,4-c]pyridin-3-yl]piperidine-2-carboxylic acid hydrochloride

[chemical structure]

Intermediate 47 was prepared similarly to Intermediate 44, using Intermediate 43 instead of Intermediate 41, to give the crude title compound as a brown solid (332 mg). LCMS (ES⁺): 357.0 [MH]+.

### Intermediate 48

### 2-{4-[1-(4-Chlorophenyl)-1H-pyrazolo[3,4-c]pyridin-3-yl]morpholin-3-yl}ethan-1-ol

[chemical structure]

Example 40 (50.0 mg, 0.13 mmol) was dissolved in DCM (1 mL), cooled to 0° C. and DIBALH (0.78 mL, 1.0 M in heptane, 0.78 mmol) was added portion-wise over 6 days. The reaction mixture was stirred for 1 week, cooled to 0° C. and quenched with water (1 mL). The reaction mixture was filtered and concentrated in vacuo to give the crude title compound as a yellow gum (51.0 mg). LCMS (ES⁺): 359.0 [MH]⁺.

### Intermediate 49

### 2-{4-[1-(4-chlorophenyl)-1H-pyrazolo[3,4-c]pyridin-3-yl]morpholin-3-yl}ethyl methanesulfonate

[chemical structure]

Intermediate 48 (50.0 mg, 0.14 mmol) was dissolved in DCM (1.5 mL), cooled to 0° C. and Et₃N (42.7 μL, 0.31 mmol) and methanesulfonyl chloride (11.9 μL, 0.15 mmol) were added. The reaction mixture was stirred for 20 h, diluted with DCM (5 mL), washed with sat aq NH₄Cl (3×5 mL) and sat aq Na₂CO₃ (5 mL), dried (MgSO₄) and concentrated in vacuo to give the title compound as a dark yellow gum (60.0 mg, 99%). LCMS (ES⁺): 437.0 [MH]⁺.

### Intermediate 50

### 1-[1-(4-Chlorophenyl)-1H-pyrazolo[3,4-c]pyridin-3-yl]piperazine dihydrochloride

[chemical structure]

Intermediate 42 (80.0 mg, 0.19 mmol) was dissolved in HCl (1.25 M in EtOH, 10 mL) and the reaction mixture was stirred for 18 h. The solvents were removed in vacuo to give the title compound as an orange solid (76.0 mg, 100%). LCMS (ES⁺): 314.0 [MH]⁺. HPLC: Rt 3.83 min, 90.0% purity.

### Intermediate 51

### tert-Butyl N-({4-[1-(4-chlorophenyl)-1H-pyrazolo[3,4-c]pyridin-3-yl]morpholin-2-yl}methyl)carbamate

[chemical structure]

Intermediate 8 (290 mg, 0.87 mmol), 4-chlorophenylboronic acid (272 mg, 1.74 mmol), anhydrous copper (II) acetate (316 mg, 1.74 mmol) and pyridine (350 uL, 4.35 mmol) were suspended in DCM (12 mL) and stirred for 24 h. The residue was dissolved in MeOH (15 mL) and purified using an SCX-2 cartridge and by column chromatography to give the title compound as a light yellow solid (100 mg, 26%). LCMS (ES⁺): 444.1 [MH]⁺. HPLC: Rt 6.01 min, 84% purity.

### Intermediate 52

### {4-[1-(4-Chlorophenyl)-1H-pyrazolo[3,4-c]pyridin-3-yl]morpholin-2-yl}methanamine dihydrochloride

[chemical structure]

Intermediate 52 was prepared similarly to Intermediate 10, using Intermediate 51 instead of Intermediate 9, to give the title compound as an orange solid (62.0 mg). LCMS (ES⁺): 344.1 [MH]⁺.

### Intermediate 53

### tert-Butyl N-[(3-aminopyridin-4-yl)methyl]-N-[2-(2-oxoimidazolidin-1-yl)ethyl]carbamate

[chemical structure]

3-Amino-pyridine-4-carbaldehyde (0.86 g, 7.04 mmol), 1-(2-aminoethyl)imidazolidin-2-one (1.00 g, 7.74 mmol) and AcOH (0.44 mL, 7.75 mmol) were dissolved in DCM (20 mL) and stirred for 1 h. NaBH(OAc)₃ (2.24 g, 10.6 mmol) was added and the reaction mixture was stirred for 3 h and diluted with DCM (10 mL) and water (20 mL). Na₂CO₃ (2.24 g, 21.1 mmol) and di-tert-butyl-dicarbonate (1.84 g, 8.45 mmol) were added and the reaction mixture was stirred for 20 h. The aqueous fraction was extracted with DCM (50 mL) and the combined organic fractions were washed with sat aq NaHCO₃ (40 mL), dried (MgSO₄) and concentrated in vacuo. The residue was purified by column chromatography to give the title compound (539 mg, 23%) as a yellow gum. LCMS (ES⁺): 336.2 [MH]⁺.

### Intermediate 54

### tert-Butyl N-[2-(2-oxoimidazolidin-1-yl)ethyl]-N-{1H-pyrazolo[3,4-c]pyridin-3-yl}carbamate

[chemical structure]

Intermediate 54 was prepared similarly to Intermediate 21, using Intermediate 53 instead of Intermediate 20, to give the title compound as a light brown solid (432 mg, 78%). LCMS (ES⁺): 347.2 [MH]⁺.

### Intermediate 55

### tert-Butyl N-[1-(4-chlorophenyl)-1H-pyrazolo[3,4-c]pyridin-3-yl]-N-[2-(2-oxoimidazolidin-1-yl)ethyl]carbamate

[chemical structure]

Intermediate 55 was prepared similarly, to Intermediate 22, using Intermediate 54 instead of Intermediate 21, to give the title compound as a yellow solid (74.0 mg, 13%). LCMS (ES⁺): 457.0 [MH]⁺. HPLC: Rt 5.33 min, 100% purity.

### Example 1

### 4-[1-(4-Chlorophenyl)-1H-pyrrolo[2,3-c]pyridin-3-yl]-1-(pyrrolidin-3-yl)piperidine

[chemical structure]

Intermediate 4 (205 mg, 0.66 mmol) and tert-butyl 3-oxopyrrolidine-1-carboxylate (229 μL, 243 mg) were dissolved in DCM (5 mL) and NaBH(OAc)₃ (348 mg, 1.64 mmol) was added. The reaction mixture was stirred for 18 h, diluted with 1 M aq Na₂CO₃ (50 mL) and extracted with DCM (2×50 mL). The combined organic fractions were dried (MgSO₄) and concentrated in vacuo. The residue was purified by column chromatography, dissolved in DCM (10 mL) and TFA (2.5 mL) and stirred for 2 h. The reaction mixture was concentrated in vacuo and the residue was dissolved in 1 M aq Na₂CO₃ (50 mL) and extracted with DCM (2×50 mL). The combined organic fractions were dried (MgSO₄) and concentrated in vacuo. The residue was purified by reverse phase HPLC to give the title compound as a colourless gum (67.0 mg, 27%). HRMS (ESI+) calcd for C22H25ClN4 381.184, found 381.1846. HPLC: Rt 3.36 min, 98% purity.

### Example 2

### 4-[1-(4-Chlorophenyl)-1H-pyrrolo[2,3-c]pyridin-3-yl]-1-(piperidin-4-yl)piperidine

[chemical structure]

Example 2 (72.0 mg, 19%) was prepared similarly to Example 1, using tert-butyl 4-oxopiperidine-1-carboxylate instead of tert-butyl 3-oxopyrrolidine-1-carboxylate. HRMS (ESI+) calcd for C23H27ClN4 395.1997, found 395.1998. HPLC: Rt 3.52 min, 99% purity.

### Example 3

### 4-[1-(4-Chlorophenyl)-1H-pyrrolo[2,3-c]pyridin-3-yl]-1-(piperidin-4-ylmethyl) piperidine

[chemical structure]

Example 3 (39.0 mg, 20%) was prepared similarly to Example 1, using tert-butyl 4-formylpiperidine-1-carboxylate instead of tert-butyl 3-oxopyrrolidine-1-carboxylate. HRMS (ESI+) calcd for C24H29ClN4 409.2153, found 409.2155. HPLC: Rt 3.55 min, 99% purity.

### Example 4

### 1-{4-[1-(4-Chlorophenyl)-1H-pyrrolo[2,3-c]pyridin-3-yl]piperidin-1-yl}-2-(piperidin-4-yl)ethan-1-one

[chemical structure]

Intermediate 4 (200 mg, 0.64 mmol), 2-{1-[(tert-butoxy)carbonyl]piperidin-4-yl}acetic acid (203 mg, 0.83 mmol), HOBt (113 mg, 0.83 mmol) and DIPEA (290 μL, 1.67 mmol) were dissolved in DMF (5 mL) and EDC (160 mg, 0.83 mmol) was added. The reaction mixture was stirred for 18 h and concentrated in vacuo. The residue was dissolved in EtOAc (25 mL) and washed with 10% aq citric acid (25 mL), 1 M aq Na₂CO₃ (25 mL) and water (25 mL), dried (MgSO₄) and concentrated in vacuo. The residue was purified by column chromatography, dissolved in DCM (10 mL) and TFA (2 mL) and stirred for 2 h. The reaction mixture was concentrated in vacuo, dissolved in 1 M aq Na₂CO₃ (25 mL) and extracted with DCM (3×25 mL). The combined organic fractions were dried (MgSO₄) and concentrated in vacuo. The residue was purified by column chromatography to give the title compound as a colourless gum (20.5 mg, 7%). HRMS (ESI+) calcd for C25H29ClN40 437.2103, found 437.21. HPLC: Rt 3.92 min, 96% purity.

### Example 5

### 1-({4-[1-(4-Chlorophenyl)-1H-pyrrolo[2,3-c]pyridin-3-yl]piperidin-1-yl}carbonyl)-4-methylpiperazine

[chemical structure]

Intermediate 4 (200 mg, 0.64 mmol), DIPEA (245 μL, 1.41 mmol) and DMAP (7.80 mg, 0.06 mmol) were dissolved in DCM (10 mL) and 4-methylpiperazine-1-carbonyl chloride hydrochloride (140 mg, 0.70 mmol) was added. The reaction mixture was stirred for 18 h, diluted with 1 M aq Na₂CO₃ (50 mL) and extracted with DCM (3×50 mL). The combined organic fractions were dried (MgSO₄) and concentrated in vacuo. The residue was purified by column chromatography and reverse phase HPLC to give the title compound as a white solid (84.0 mg, 30%). HRMS (ESI+) calcd for C24H28ClN5O 438.2055, found 438.2057. HPLC: Rt 3.92 min, 100% purity.

### Example 6

### 4-[1-(4-Chlorophenyl)-1H-pyrrolo[2,3-c]pyridin-3-yl]-N-(piperidin-4-ylmethyl)piperidine-1-carboxamide

[chemical structure]

CDI (187 mg, 1.15 mmol) was dissolved in DCM (10 mL), a solution of tert-butyl 4-(aminomethyl)piperidine-1-carboxylate (247 mg, 1.15 Mmol) and DIPEA (251 μL, 1.15 mmol) in DCM (2 mL) was added and the reaction mixture was stirred for 18 h. A solution of Intermediate 4 (300 mg, 0.96 mmol) and DIPEA (251 μL, 1.15 mmol) in DCM (2 mL) was added and the reaction mixture was stirred for 24 h, mixed with 1 M aq Na₂CO₃ (50 mL) and extracted with DCM (3×50 mL). The combined organic fractions were dried (MgSO₄) and concentrated in vacuo. The residue was purified by column chromatography, dissolved in DCM (10 mL) and TFA (2.5 mL) and stirred for 1 h. The reaction mixture was concentrated in vacuo, dissolved in 1 M aq Na₂CO₃ (50 mL) and extracted with DCM (3×50 mL). The combined organic fractions were dried (MgSO₄) and concentrated in vacuo. The residue was purified by reverse phase HPLC to give the title compound as a colourless gum (52.0 mg, 12%). HRMS (ESI+) calcd for C25H30ClN5O 452.2212, found 452.2213. HPLC: Rt 3.92 min, 100% purity.

### Examples 7-26

Examples 7-26 were prepared similarly to Example 6, by CDI (or triphosgene) coupling of Intermediate 4 with the appropriate amine, and subsequent Boc deprotection (where required); see Table 4 below.

TABLE 4
Urea couplings with Intermediate 4 and subsequent Boc deprotection (where required)

[chemical structure]

[chemical structure]

[chemical structure]

| Ex | Structure | Name | Yield | LCMS, HPLC |
|---|---|---|---|---|
| 7 | [chemical structure] | 4-[1-(4- Chlorophenyl)- 1H-pyrrolo[2,3- c]pyridin-3- yl]-N- (piperidin-4-yl) piperidine-1- carboxamide | 80.0 mg 19% | HRMS (ESI+) calcd for C24H28ClN5O 438.2055, found 438.2057. HPLC: Rt 3.91 min, 100% purity. |
| 8 | [chemical structure] | 4-[1-(4- Chlorophenyl)- 1H-pyrrolo[2,3- c]pyridin-3-yl]- N-(1- methylpiperidin- 4-yl)piperidine- 1-carboxamide | 91.0 mg 21% | HRMS (ESI+) calcd for C25H30ClN5O 452.2212, found 452.2214. HPLC: Rt 4.01 min, 99% purity. |
| 9 | [chemical structure] | 4-[1-(4- Chlorophenyl)- 1H-pyrrolo[2,3- c]pyridin-3-yl]- N-[1- (propan-2-yl) piperidin-4-yl] piperidine-1- carboxamide | 125 mg 27% | HRMS (ESI+) calcd for C27H34ClN5O 480.2525, found 480.2521. HPLC: Rt 4.15 min, 99% purity. |
| 10 | [chemical structure] | N-(1- Acetylpiperidin- 4-yl)-4-[1-(4- chlorophenyl)- 1H-pyrrolo [2,3-c]pyridin- 3-yl]piperidine- 1-carboxamide | 184 mg 40% | HRMS (ESI+) calcd for C26H30ClN5O2 480.2161, found 480.2159. HPLC: Rt 4.50 min, 100% purity. |
| 11 | [chemical structure] | 4-[1-(4- Chlorophenyl)- 1H-pyrrolo[2,3- c]pyridin-3- yl]-N-[(1- methylpiperidin- 4-yl)methyl] piperidine- 1-carboxamide | 96.0 mg 21% | HRMS (ESI+) calcd for C26H32ClN5O 466.2368, found 466.2379. HPLC: Rt 4.02 min, 99% purity. |
| 12 | [chemical structure] | 4-[1-(4- Chlorophenyl)- 1H-pyrrolo[2,3- c]pyridin-3- yl]-N-[(1- ethylpiperidin- 4-yl)methyl] piperidine- 1-carboxamide | 122 mg 26% | HRMS (ESI+) calcd for C27H34ClN5O 480.2525, found 480.2521. HPLC: Rt 4.10 min, 99% purity. |
| 13 | [chemical structure] | 4-[1-(4- Chlorophenyl)- 1H-pyrrolo[2,3- c]pyridin-3- yl]-N- methyl-N-[(1- methylpiperidin- 4-yl)methyl] piperidine-1- carboxamide; formic acid | 4.57 mg 1% | LCMS (ES+): 480.1 [MH]⁺. HPLC: Rt 4.22 min, 96% purity. |
| 14 | [chemical structure] | N-{[1- (Carbamoyl- methyl) piperidin-4-yl] methyl}-4-[1-(4- chlorophenyl)- 1H-pyrrolo[2,3- c]pyridin-3-yl] piperidine-1- carboxamide; formic acid | 32.8 mg 11% | HRMS (ESI+) calcd for C27H33ClN6O2 509.2426, found 509.2418. HPLC: Rt 3.93 min, 100% purity. |
| 15 | [chemical structure] | 4-[1-(4- Chlorophenyl)- 1H-pyrrolo[2,3- c]pyridin-3-yl]- N-methyl-N-{[1- (propan-2-yl) piperidin-4-yl] methyl} piperidine- 1-carboxamide | 28.2 mg 6% | HRMS (ESI+) calcd for C29H38ClN5O 508.2838, found 508.2835. HPLC: Rt 4.39 min, 100% purity. |
| 16 | [chemical structure] | 1-({4-[1-(4- Chlorophenyl)- 1H-pyrrolo[2,3- c]pyridin-3-yl] piperidin-1-yl} carbonyl)-4- cyclopropyl- piperazine | 49.2 mg 11% | HRMS (ESI+) calcd for C26H30ClN5O 464.2212, found 464.2208. HPLC: Rt 4.11 min, 100% purity. |
| 17 | [chemical structure] | 1-({4-[1-(4- Chlorophenyl)- 1H-pyrrolo[2,3- c]pyridin-3-yl] piperidin-1-yl} carbonyl)-4- (propan-2- yl)piperazine | 38.0 mg 11% | HRMS (ESI+) calcd for C26H32ClN5O 466.2368, found 466.2359. HPLC: Rt 4.19 min, 100% purity. |
| 18 | [chemical structure] | 1-({4-[1-(4- Chlorophenyl)- 1H-pyrrolo[2,3- c]pyridin-3-yl] piperidin-1- yl}carbonyl)-4- (2- methoxyethyl) piperazine | 69.5 mg 15% | HRMS (ESI+) calcd for C26H32ClN5O2 482.2317, found 482.2314. HPLC: Rt 4.09 min, 99% purity. |
| 19 | [chemical structure] | (3S)-1-({4-[1-(4- Chlorophenyl)- 1H- pyrrolo[2,3-c] pyridin- 3-yl]piperidin-1- yl}carbonyl)-3- (propan-2-yl) piperazine | 58.0 mg 16% | HRMS (ESI+) calcd for C26H32ClN5O 466.2368, found 466.2363. HPLC: Rt 4.17 min, 100% purity. |
| 20 | [chemical structure] | 4-[1-(4- Chlorophenyl)- 1H-pyrrolo[2,3- c]pyridin-3- yl]-N- (morpholin-2- ylmethyl) pipendine-1- carboxamide | 120 mg 27% | HRMS (ESI+) calcd for C24H28ClN5O2 454.2004, found 454.2004. HPLC: Rt 3.93 min, 100% purity. |
| 21 | [chemical structure] | 4-[1-(4- Chlorophenyl)- 1H-pyrrolo[2,3- c]pyridin-3- yl]-N- [(1,4-dimethyl- piperazin- 2-yl)methyl] piperidine-1- carboxamide | 123 mg 29% | HRMS (ESI+) calcd for C26H33ClN6O 481.2477, found 481.2475. HPLC: Rt 3.79 min, 100% purity. |
| 22 | [chemical structure] | 4-[1-(4- Chlorophenyl)- 1H-pyrrolo[2,3- c]pyridin-3-yl]- N-[2- (morpholin-4-yl) ethyl]piperidine- 1-carboxamide | 91.0 mg 20% | HRMS (ESI+) calcd for C25H30ClN5O2 468.2161, found 468.2158. HPLC: Rt 4.34 min, 100% purity. |
| 23 | [chemical structure] | 4-[1-(4- Chlorophenyl)- 1H-pyrrolo[2,3- c]pyridin-3- yl]-N- [2-(piperazin-1- yl)ethyl] piperidine-1- carboxamide | 110 mg 24% | LCMS (ES⁺): 467.0 [MH]⁺. HPLC: Rt 3.66 min, 99% purity. |
| 24 | [chemical structure] | 4-[1-(4- Chlorophenyl)- 1H-pyrrolo[2,3- c]pyridin-3-yl]- N-[2-(1- methylpiperidin- 4-yl)ethyl] piperidine- 1-carboxamide | 83.2 mg 18% | HRMS (ESI+) calcd for C27H34ClN5O 480.2525, found 480.2525. HPLC: Rt 4.13 min, 99% purity. |
| 25 | [chemical structure] | 4-[1-(4- Chlorophenyl)- 1H-pyrrolo[2,3- c]pyridin-3- yl]-N- [2-(4- methylpiperazin- 1-yl)ethyl] piperidine- 1-carboxamide | 72.4 mg 16% | HRMS (ESI+) calcd for C26H33ClN6O 481.2477, found 481.2474. HPLC: Rt 3.76 min, 100% purity. |
| 26 | [chemical structure] | 4-(1-(4- Chlorophenyl)- 1H-pyrrolo[2,3- c]pyridin-3-yl]- N-[3- (morpholin-4-yl) propyl] piperidine- 1-carboxamide | 91.0 mg 20% | LCMS (ES⁺): 482.0 [MH]⁺. HPLC: Rt 4.01 min, 100% purity. |

### Example 27

### 4-[1-(4-Chlorophenyl)-1H-pyrrolo[2,3-c]pyridin-3-yl]-N-{[1-(propan-2-yl)piperidin-4-yl]methyl}piperidine-1-carboxamide; formic acid

[chemical structure]

Intermediate 5 (250 mg, 0.55 mmol) and acetone (81.1 μL, 1.11 mmol) were dissolved in DCM (200 mL) and stirred for 1 h. NaBH(OAc)₃ (293 mg, 1.38 mmol) was added and the reaction mixture was stirred for 18 h, diluted with 1 M aq Na₂CO₃ (50 mL) and extracted with DCM (3×50 mL). The combined organic fractions were dried (MgSO₄) and concentrated in vacuo. The residue was purified by reverse phase HPLC (formic acid buffered) to give the title compound as a white solid (20.5 mg, 7%). LCMS (ES⁺): 494.1 [MH]⁺. HPLC: Rt 4.16 min, 98% purity.

### Example 28

### 4-[1-(4-Chlorophenyl)-1H-pyrrolo[2,3-c]pyridin-3-yl]-N-{[1-(2-methoxyethyl)piperidin-4-yl]methyl}piperidine-1-carboxamide; formic acid

[chemical structure]

Intermediate 5 (250 mg, 0.55 mmol) was dissolved in MeCN (3 mL) and K₂CO₃ (229 mg, 1.66 mmol) and 1-bromo-2-methoxyethane (52.0 μL, 0.55 mmol) were added. The reaction mixture was heated in a microwave reactor at 100° C. for 30 min, diluted with water (50 mL) and extracted with DCM (3×50 mL). The combined organic fractions were dried (MgSO₄) and concentrated in vacuo. The residue was purified by reverse phase HPLC (formic acid buffered) to give the title compound as a white solid (11.3 mg, 4%). LCMS (ES⁺): 510.0 [MH]⁺. HPLC: Rt 4.08 min, 94%, purity.

### Example 29

### N-[3-({4-[1-(4-Chlorophenyl)-1H-pyrrolo[2,3-c]pyridin-3-yl]piperidin-1-yl}carbonylamino)propyl]acetamide

[chemical structure]

Intermediate 6 (64.0 mg, 0.16 mmol) was dissolved in DCM (5 mL) and Et₃N (22.8 μL, 0.16 mmol) and Ac₂O (15.4 μL, 0.16 mmol) were added. The reaction mixture was stirred for 18 h then diluted with sat aq Na₂CO₃ (25 mL) and extracted with DCM (3×25 mL). The combined organic fractions were dried (MgSO₄) and concentrated in vacuo. The residue was purified by reverse phase HPLC to give the title compound as a white solid (63.0 mg, 89%). HRMS (ESI+) calcd for C24H28ClN5O2 454.2004, found 454.2004. HPLC: Rt 4.34 min, 98% purity.

### Example 30

### Propan-2-yl N-({4-[1-(4-methylphenyl)-1H-pyrazolo[3,4-c]pyridin-3-yl]morpholin-2-yl}methyl)carbamate

[chemical structure]

Intermediate 10 (100 mg, 0.25 mmol) and isopropyl chloroformate (278 μL, 0.28 mmol) were added to a mixture of DCM (2 mL) and sat aq K₂CO₃ (3.5 mL) and the reaction mixture was stirred for 1 h. The aq fraction was extracted with DCM (2×50 mL) and the combined organic fractions were dried (MgSO₄) and concentrated in vacuo. The residue was purified by column chromatography to give the title compound as a white solid (18.9 mg, 18%). HRMS (ESI+) calcd for C₂₂H₂₇N₅O₃ 410.2187, found 410.2188. HPLC: Rt 5.31 min, 98% purity.

### Example 31

### 3-Cyclopropyl-1-({4-[1-(4-methylphenyl)-1H-pyrazolo[3,4-c]pyridin-3-yl]morpholin-2-yl}methyl)urea

[chemical structure]

Cyclopropylamine (17.5 μL, 0.25 mmol) and CDI (40.9 mg, 0.25 mmol) were dissolved in DMF (1 mL) and stirred for 6 h. A solution of Intermediate 10 (100 mg, 0.25 mmol) in DMF (1 mL) and DIPEA (92.6 μL, 0.56 mmol) were added and the reaction mixture was stirred at 50° C. overnight. The solvents were removed in vacuo and the residue was dissolved in DCM (10 mL) and washed with sat aq Na₂CO₃ (5 mL). The aq fraction was extracted with DCM (10 mL) and the combined organic fractions were dried (MgSO₄) and concentrated in vacuo. The residue was purified by column chromatography to give the title compound as a light yellow solid (32.7 mg, 32%). HRMS (ESI+) calcd for C₂₂H₂₆N₆O₂ 407.2190, found 407.2192. HPLC: Rt 4.59 min, 98% purity.

### Example 32

### 2-({4-[1-(4-Methylphenyl)-1H-pyrazolo[3,4-c]pyridin-3-yl]morpholin-2-yl}methoxy)ethan-1-amine

[chemical structure]

tert-Butyl N-(2-hydroxyethyl)carbamate (159 μL, 1.03 mmol) was dissolved in DMF (0.50 mL), NaH (49.2 mg, 60% dispersion in mineral oil, 1.03 mmol) was added and the reaction mixture was stirred at 50° C. for 30 min. A solution of Intermediate 18 (92.0 mg, 0.23 mmol) in DMF (0.5 mL) was added drop-wise and the reaction mixture was stirred at 65° C. for 18 h and at 80° C. for 4 h. The reaction mixture was cooled to 0° C. and quenched with water (1 mL). The reaction mixture was concentrated in vacuo and the residue was purified by column chromatography to give the title compound as an orange gum (3.05 mg, 4%). HRMS (ESI+) calcd for C20H25N5O2 368.2081, found 368.2084. HPLC: Rt 3.96 min, 97.4% purity.

### Example 33

### (2-Aminoethyl)({4-[1-(4-methylphenyl)-1H-pyrazolo[3,4-c]pyridin-3-yl]morpholin-2-yl}methyl)amine trihydrochloride

[chemical structure]

Intermediate 18 (92.0 mg, 0.23 mmol), tert-butyl N-(2-aminoethyl)carbamate (110 mg, 0.69 mmol), K₂CO₃ (126 mg, 0.91 mmol) and Cs₂CO₃ (100 mg, 0.31 mmol) were suspended in MeCN (2 mL) and DMF (1 mL) and the reaction mixture was heated at 90° C. for 20 h. The reaction mixture was concentrated in vacuo and the residue was purified by column chromatography, dissolved in 1.25 M HCl in EtOH (2.5 mL) and stirred for 18 h. The reaction mixture was concentrated in vacuo to give the title compound as a dark yellow solid (1.52 mg, 30%). LCMS (ES⁺): 367.0 [MH]⁺. HPLC: Rt 3.57 min, 98.2% purity.

### Example 34

### 4-[1-(4-Methylphenyl)-1H-pyrazolo[3,4-c]pyridin-3-yl]-2-(morpholin-4-ylmethyl)morpholine

[chemical structure]

Intermediate 18 (92.0 mg, 0.23 mmol), morpholine (60.0 μL, 0.69 mmol), K₂CO₃ (126 mg, 0.91 mmol) and Cs₂CO₃ (100 mg, 0.31 mmol) were suspended in MeCN (2 mL) and DMF (1 mL) and the reaction mixture was heated at 90° C. for 20 h. The reaction mixture was concentrated in vacuo and the residue was purified by column chromatography to give the title compound as a yellow solid (7.46 mg, 8%). HRMS (ESI+) calcd for C22H27N5O2 394.2238, found 394.2239. HPLC: Rt 3.94 min, 94.5% purity.

### Example 35

### 4-[1-(4-Chlorophenyl)-1H-pyrazolo[3,4-c]pyridin-3-yl]-2-[(4-methylpiperazin-1-yl)methyl]morpholine

[chemical structure]

Example 35′ (1.23 mg, 2%) was prepared similarly to Example 34, using Intermediate 19 instead of Intermediate 18 and 1-methylpiperazine instead of morpholine. LCMS (ES+): 427.0 [MH]⁺. HPLC: Rt 3.85 min, 97.1% purity.

### Example 36

### 4-[1-(4-Chlorophenyl)-1H-pyrazolo[3,4-c]pyridin-3-yl]-2-(piperazin-1-ylmethyl) morpholine trihydrochloride

[chemical structure]

Intermediate 22 (75.0 mg, 0.15 mmol) was dissolved in 1.25 M HCl in EtOH (15 mL) and the reaction mixture was stirred for 18 h and concentrated in vacuo to give the title compound as an orange solid (74.6 mg, 97%). HRMS (ESI+) calcd for C21H25ClN6O 413.1851, found 413.1853. HPLC: Rt 3.76 min, 97.8% purity.

### Example 37

### 3-Aminopropyl 4-({4-[1-(4-chlorophenyl)-1H-pyrazolo[3,4-c]pyridin-3-yl]morpholin-2-yl}methyl)piperazine-1-carboxylate trihydrochloride

[chemical structure]

Triphosgene (14.2 mg, 0.05 mmol) was dissolved in DCM (1 mL) and a solution of tert-butyl N-(3-hydroxypropyl)carbamate (25.2 mg, 0.14 mmol) and DIPEA (25.0 μL, 0.14 mmol) in DCM (1 mL) was added. The reaction mixture was stirred for 1 h, a solution of Example 36 (50.0 mg, 0.10 mmol) and DIPEA (25.0 μL, 0.14 mmol) in DCM (1 mL) was added and the reaction mixture was stirred for 4 d. The reaction mixture was diluted with DCM (10 mL) and washed with sat aq NH₄Cl (5×10 mL). The organic fraction was dried (MgSO₄) and concentrated in vacuo. The residue was purified by reverse phase chromatography, dissolved in 1.25 M HCl in EtOH (5 mL) and stirred for 16 h. The solvents were removed in vacuo to give the title compound as an orange solid (19.5 mg, 35%). HRMS (ESI+) calcd for C25H32ClN7O3 514.2328, found 514.2326. HPLC: Rt 3.79 min, 98.3% purity.

### Example 38

### N-(3-Aminopropyl)-4-({4-[1-(4-chlorophenyl)-1H-pyrazolo[3,4-c]pyridin-3-yl]morpholin-2-yl}methyl)piperazine-1-carboxamide tri hydrochloride

[chemical structure]

Example 38 (24.6 mg, 41%) was prepared similarly to Example 37, using tert-butyl N-(3-aminopropyl)carbamate instead of tert-butyl N-(3-hydroxypropyl)carbamate. HRMS (ESI+) calcd for C25H33ClN8O2 513.2488, found 513.2486. HPLC: Rt 3.73 min, 100% purity.

### Example 39

### 4-({4-[1-(4-Chlorophenyl)-1H-pyrazolo[3,4-c]pyridin-3-yl]morpholin-2-yl}methyl)-N-ethylpiperazine-1-carboxamide

[chemical structure]

Triphosgene (14.2 mg, 0.05 mmol) was dissolved in DCM (1 mL) and a solution of ethylamine (9.52 μL, 0.14 mmol) and DIPEA (25.0 μL, 0.14 mmol) in DCM (1 mL) was added. The reaction mixture was stirred for 1 h and a solution of Example 36 (50.0 mg, 0.10 mmol) and DIPEA (25.0 μL, 0.14 mmol) in DCM (1 mL) was added. The reaction mixture was stirred for 4 d, diluted with DCM (10 mL) and washed with sat aq NH₄Cl (5×10 mL). The organic fraction was dried (MgSO₄) concentrated in vacuo. The residue was purified by reverse phase chromatography to give the title compound as a pale yellow solid (18.6 mg, 40%). HRMS (ESI+) calcd for C24H30ClN7O2 484.2222, found 484.2219. HPLC: Rt 4.15 min, 99.3% purity.

### Example 40

Methyl 2-{4-[1-(4-chlorophenyl)-1H-pyrazolo[3,4-c]pyridin-3-yl]morpholin-3-yl}acetate

[chemical structure]

Intermediate 33 (1.64 g, 5.94 mmol), 4-chlorophenylboronic acid (1.86 g, 11.9 mmol), Cu(OAc)₂ (2.16 g, 11.9 mmol) and pyridine (2.39 mL, 29.7 mmol) were suspended in DCE (41 mL) and stirred overnight. The reaction mixture was purified by column chromatography to give the title compound as a yellow gum (866 mg, 38%). HRMS (ESI+) calcd for C19H19ClN4O3 387.1218, found 387.1218. HPLC: Rt 5.32 min, 100% purity.

### Example 41

### 4-[1-(4-Chlorophenyl)-1H-pyrazolo[3,4-c]pyridin-3-yl]-3-(morpholin-4-ylmethyl)morpholine

[chemical structure]

Example 41 was prepared similarly to Example 40, by N-arylation of Intermediate 38 to give the title compound as a yellow gum (6.00 mg, 5%). HRMS (ESI+) calcd for C21H24ClN5O2 414.1691, found 414.1693. HPLC: Rt 4.04 min, 97.6% purity.

### Example 42

### 4-[1-(4-Chlorophenyl)-1H-pyrazolo[3,4-c]pyridin-3-yl]-3-[2-(4-methyl piperazin-1-yl)ethyl]morpholine

[chemical structure]

Intermediate 49 (60.0 mg, 0.14 mmol), 1-methyl-piperazine (45.7 μL, 0.41 mmol) and K₂CO₃ (75.9 mg, 0.55 mmol) were suspended in MeCN (1 mL) and the reaction mixture was heated at 50° C. for 4 h and at 75° C. for 4 h. The reaction mixture was filtered, concentrated in vacuo and the residue was purified by column chromatography to give the title compound as a dark yellow gum (3.47 mg, 6%). HRMS (ESI+) calcd for C23H29ClN6O 441.2164, found 441.2164. HPLC: Rt 3.89 min, 99.1% purity.

### Example 43

### 1-[1-(4-Chlorophenyl)-1H-pyrazolo[3,4-c]pyridin-3-yl]-N-[(1-methylpiperidin-4-yl)methyl]piperidine-2-carboxamide

[chemical structure]

Intermediate 47 (66.0 mg, 0.17 mmol) was dissolved in DMF (2 mL), cooled to 0° C. and HBTU (63.3 mg, 0.17 mmol), (1-methyl-4-piperidinyl)methanamine (25.7 mg, 0.20 mmol) and DIPEA (58.2 μL, 0.33 mmol) were added. The reaction mixture was stirred at 0° C. for 1 h and at RT for 18 h, diluted with DCM (10 mL) and washed with sat aq NH₄Cl (3×5 mL). The organic fraction was dried (MgSO₄) and concentrated in vacuo. The residue was purified by column chromatography to give the title compound as a yellow solid (8.62 mg, 11%). LCMS (ES⁺): 467.0 [MH]⁺. HPLC: Rt 4.19 min, 100% purity.

### Example 44

### 1-(4-Chlorophenyl)-N-[2-(morpholin-4-yl)ethyl]-1H-pyrazolo[3,4-c]pyridin-3-amine

[chemical structure]

Example 44 was prepared similarly to Example 40, by N-arylation of Intermediate 35 and subsequent Boc deprotection (HCl in EtOH) to give the title compound as a pale brown gum (2.27 mg, 1%). HRMS (ESI+) calcd for C18H20ClN5O 358.1429, found 358.1434. HPLC: Rt 4.00 min, 97% purity.

### Example 45

### 1-(4-Chlorophenyl)-N-[2-(piperazin-1-yl)ethyl]-1H-pyrazolo[3,4-c]pyridin-3-amine

[chemical structure]

Example 45 was prepared similarly to Example 40, by N-arylation of Intermediate 36 and subsequent Boc deprotection (HCl in Et₂O) to give the title compound as a pale green gum (9.98 mg, 10%). HRMS (ESI+) calcd for C18H21ClN6 357.1589, found 357.1592. HPLC: Rt 3.57 min, 99.5% purity.

### Example 46

### 1-(4-Chlorophenyl)-N-[2-(4-methylpiperazin-1-yl)ethyl]-1H-pyrazolo[3,4-c]pyridin-3-amine

[chemical structure]

Example 45 (24.6 mg, 0.07 mmol) was dissolved in MeOH (2 mL), formaldehyde (55.9 mg, 37% in water, 0.69 mmol) was added and the reaction mixture was stirred for 30 min. NaBH(OAc)₃ (17.5 mg, 0.08 mmol) was added and the reaction mixture was stirred overnight and concentrated in vacuo. The residue was purified by reverse phase HPLC to give the title compound as a pale green gum (16.0 mg, 63%). HRMS (ESI+) calcd for C19H23ClN6 371.1745, found 371.1751. HPLC: Rt 3.54 min, 100% purity.

### Example 47

### 1-[1-(4-Chlorophenyl)-1H-pyrazolo[3,4-c]pyridin-3-yl]-N-(piperidin-4-ylmethyl)piperidine-4-carboxamide dihydrochloride

[chemical structure]

Intermediate 44 (200 mg, 0.51 mmol) was dissolved in DMF (2 mL) and HBTU (231 mg, 0.61 mmol) was added. The reaction mixture was stirred for 30 min, tert-butyl 4-(aminomethyl)piperidine-1-carboxylate (131 mg, 0.61 mmol) and DIPEA (266 μL, 1.53 mmol) were added and the reaction mixture was stirred overnight. The solvents were removed in vacuo and the residue was diluted with EtOAc (25 mL), washed with sat aq NH₄Cl (4×25 mL), dried (MgSO₄) and concentrated in vacuo. The residue was purified by column chromatography and half of the product was dissolved in 1.25 M HCl in EtOH (10 mL) and stirred for 18 h. The reaction mixture was concentrated in vacuo to give the title compound as an orange solid (77.9 mg, 58%). HRMS (ESI+) calcd for C24H29ClN6O 453.2164, found 453.2163. HPLC: Rt 4.07 min, 98.1% purity.

### Example 48

### 4-({1-[1-(4-Chlorophenyl)-1H-pyrazolo[3,4-c]pyridin-3-yl]piperidin-4-yl}methyl) morpholine dihydrochloride

[chemical structure]

Intermediate 45 (100 mg, 0.23 mmol) was dissolved in THF (1 mL) and 1.0 M BH₃ in THF (1.88 mL, 1.88 mmol) was added portion-wise with heating at 67° C. for 2 d. The reaction mixture was cooled to 0° C., quenched with cold water (2 mL) and concentrated in vacuo. The residue was purified by column chromatography, dissolved in 1.25 M HCl in EtOH (5 mL), stirred for 4 h and concentrated in vacuo to give the title compound as an orange solid (5.68 mg, 5%). HRMS (ESI+) calcd for C22H26ClN5O 412.1899, found 412.1896. HPLC: Rt 4.13 min, 98.1% purity.

### Examples 49-50

Examples 49-50 were prepared similarly to Example 48, by borane reduction of Intermediate 46 and Boc protected Example 47, and subsequent Boc deprotection; see Table 5 below.

TABLE 5
Borane reduction and subsequent Boc deprotection

[chemical structure]

[chemical structure]

| | | | Int/ | |
|---|---|---|---|---|
| Ex | Structure | Name | Yield | LCMS, HPLC |
| 49 | [chemical structure] | 1-({1-[1-(4- Chlorophenyl)- 1H-pyrazolo[3,4-c] pyridin-3-yl]piperidin- 4-yl}methyl)piperazine | Int 46 3.35 mg 3% | HRMS (ESI+) calcd for C22H27ClN6 411.2058, found 411.2061. HPLC: Rt 3.75 min, 100% purity. |
| 50 | [chemical structure] | ({1-[1-(4-Chlorophenyl)- 1H-pyrazolo[3,4-c] pyridin-3-yl]piperidin-4- yl}methyl)(piperidin-4- ylmethyl)amine | Ex 47 (Boc) 9.13 mg 9% | HRMS (ESI+) calcd for C24H31ClN6 439.2371, found 439.2372. HPLC: Rt 3.74 min, 99.6% purity. |

### Example 51

### 4-[1-(4-Chlorophenyl)-1H-pyrazolo[3,4-c]pyridin-3-yl]-N-[(1-methylpiperidin-4-yl)methyl]piperazine-1-carboxamide

[chemical structure]

Triphosgene (14.2 mg, 0.05 mmol) was dissolved in DCM (1 mL) and a solution of (1-methyl-4-piperidinyl)methanamine (18.4 mg, 0.14 mmol) and DIPEA (25.0 μL, 0.14 mmol) in DCM (1 mL) was added. The reaction mixture was stirred for 1 h and a solution of Intermediate 50 (37.0 mg, 0.10 mmol) and DIPEA (25.0 μL, 0.14 mmol) in DCM (1 mL) was added. The reaction mixture was stirred for 18 h, diluted with DCM (10 mL), washed with sat aq NH₄Cl (5×10 mL), dried (MgSO₄) and concentrated in vacuo. The residue was purified by reverse phase chromatography to give the title compound as a yellow solid (17.0 mg, 38%). LCMS (ES⁺): 468.0 [MH]⁺. HPLC: Rt 4.13 min, 98.7% purity.

### Example 52

### 1-[1-(4-Methyl phenyl)-1H-pyrazolo[3,4-c]pyridin-3-yl]piperidin-4-yl acetate

[chemical structure]

Intermediate 37 (618 mg, 2.37 mmol) was dissolved in DMF (3.5 mL) and 1-methyl-4-iodo-benzene (621 mg, 2.85 mmol), N,N′-dimethylethylenediamine (51.1 μL, 0.42 mmol), K₃PO₄ (1.06 g, 4.99 mmol) and CuI (45.2 mg, 0.24 mmol) were added. The reaction mixture was heated using a microwave reactor at 60° C. for 10 min. The reaction mixture was concentrated in vacuo and the residue was purified by column chromatography to give the title compound as a yellow gum (248 mg, 30%). HRMS (ESI+) calcd for C20H22N4O2 351.1816, found 351.1819. HPLC: Rt 5.44 min, 100% purity.

### Example 53

### 2-{4-[1-(4-Chlorophenyl)-1H-pyrazolo[3,4-c]pyridin-3-yl]morpholin-3-yl}acetic acid hydrochloride

[chemical structure]

Example 40 (1.00 g, 2.84 mmol) was dissolved in 1:1 THF/water (16 mL), LiOH.H₂O (262 mg, 6.24 mmol) was added and the reaction mixture was stirred for 3 h. The THF was removed in vacuo and the reaction mixture was acidified to pH 1 with 1 M aq HCl (5 mL). The precipitate was collected by filtration and washed with water to give the title compound as an orange solid (28.3 mg, 3%). HRMS (ESI+) calcd for C18H17ClN4O3 373.1062, found 373.1062. HPLC: Rt 4.40 min, 97% purity.

### Example 54

### N-(2-Aminoethyl)-2-{4-[1-(4-chlorophenyl)-1H-pyrazolo[3,4-c]pyridin-3-yl]morpholin-3-yl}acetamide dihydrochloride

[chemical structure]

Example 53 (180 mg, 0.44 mmol) was dissolved in DMF (2.1 mL) and cooled to 0° C., and HBTU (167 mg, 0.44 mmol), tert-butyl N-(2-aminoethyl)carbamate (84.6 mg, 0.53 mmol) and DIPEA (76.6 μL, 0.44 mmol) were added. The reaction mixture was stirred at 0° C. for 2.5 h and purified by column chromatography. The residue was dissolved in 1.25 M HCl in EtOH (2.5 mL) and stirred for 2 h. The reaction mixture was concentrated in vacuo to give the title compound as an orange solid (46.4 mg, 22%). HRMS (ESI+) calcd for C20H23ClN6O2 415.1644, found 415.1638. HPLC: Rt 3.97 min, 99% purity.

### Examples 55-58

Examples 55-58 were prepared similarly to Example 54, by amide coupling to Example 53 (no HCl salt formation step); see Table 6 below.

TABLE 6
Amide couplings to Example 53

[chemical structure]

[chemical structure]

| | | | | LCMS, |
|---|---|---|---|---|
| Ex | Structure | Name | Yield | HPLC |
| 55 | [chemical structure] | 2-{4-[1-(4- Chlorophenyl)- 1H-pyrazolo[3,4- c]pyridin-3-yl] morpholin-3-yl}- 1-(4- methylpiperazin- 1-yl)ethan-1-one | 7.95 mg 12% | LCMS (ES+): 454.9 [MH]⁺. HPLC: Rt 4.09 min, 99.8% purity. |
| 56 | [chemical structure] | 2-{4-[1-(4- Chlorophenyl)- 1H-pyrazolo[3,4- c]pyridin-3-yl] morpholin- 3-yl}-1-[(3S)-3- (dimethylamino) pyrrolidin-1-yl] ethan-1-one | 30.9 mg 22% | LCMS (ES+): 468.9 [MH]⁺. HPLC: Rt 4.11 min, 98.6% purity. |
| 57 | [chemical structure] | 2-{4-[1-(4- Chlorophenyl)- 1H-pyrazolo[3,4- c]pyridin-3-yl] morpholin-3-yl)- N-(1- methylpiperidin- 4-yl)acetamide | 31.8 mg 46% | LCMS (ES+): 468.9 [MH]⁺. HPLC: Rt 4.06 min, 98.8% purity. |
| 58 | [chemical structure] | 2- {4-[1-(4- Chlorophenyl)- 1H-pyrazolo[3,4- c]pyridin-3-yl] morpholin-3-yl}- N-[(1- methylpiperidin- 4-yl)methyl] acetamide | 17.9 mg 25% | LCMS (ES+): 482.9 [MH]⁺. HPLC: Rt 4.10 min, 98.1% purity. |

### Example 59

### ({4-[1-(4-Chlorophenyl)-1H-pyrazolo[3,4-c]pyridin-3-yl]morpholin-2-yl}methyl)urea

[chemical structure]

Intermediate 52 (50.0 mg, 0.12 mmol) and DIPEA (41.8 uL, 0.24 mmol) were dissolved in DMF (2 mL). Trimethylsilyl isocyanate (29.2 uL, 0.22 mmol) was added and the reaction mixture was stirred for 48 h. The reaction mixture was concentrated in vacuo and the residue was dissolved in EtOAc (10 mL) and washed with water (2×5 mL). The organic fraction was concentrated in vacuo and purified by reverse phase HPLC to give the title compound as a white solid (16.5 mg, 35%). LCMS (ES⁺): 387.2 [MH]⁺. HPLC: Rt 4.40 min, 96.8% purity.

### Example 60

### 1-({4-[1-(4-Chlorophenyl)-1H-pyrazolo[3,4-c]pyridin-3-yl]morpholin-2-yl}methyl)-3-methylurea

[chemical structure]

Intermediate 52 (50.0 mg, 0.12 mmol) and DIPEA (62.7 uL, 0.36 mmol) were dissolved in THF (2 mL). N-methylcarbamoyl chloride (12.3 mg, 0.132 mmol) was added and the reaction mixture was stirred overnight. The reaction mixture was concentrated in vacuo and the residue was dissolved in EtOAc (10 mL) and washed with water (2×5 mL). The organic fraction was concentrated in vacuo and purified by reverse phase HPLC to give the title compound as an off white solid (11.1 mg, 23%). LCMS (ES⁺): 401.1 [MH]⁺. HPLC: Rt 4.55 min, 99.7% purity.

### Example 61

### 4-[1-(4-Chlorophenyl)-1H-pyrrolo[2,3-c]pyridin-3-yl]-N-(2H-1,2,3,4-tetrazol-5-ylmethyl)piperidine-1-carboxamide; trifluoroacetic acid

[chemical structure]

CDI (172 mg, 1.06 mmol) was dissolved in DCM (10 mL) and a suspension of 1H-1,2,3,4-tetrazol-5-ylmethanamine hydrochloride (143 mg, 1.06 mmol) and DIPEA (368 uL, 2.12 mmol) in DCM was added. The reaction mixture was stirred for 6 h and a solution of Intermediate 4 (300 mg, 0.96 mmol) and DIPEA (368 uL, 2.12 mmol) in DCM (2 mL) was added. The reaction mixture was stirred for 3 d and concentrated in vacuo. The residue was purified by reverse phase HPLC to give the title compound as a white solid (134 mg, 25%). LCMS (ES⁺): 437.0 [MH]⁺. HPLC: Rt 4.29 min, 100% purity.

### Example 62

### 4-[1-(4-Chlorophenyl)-1H-pyrrolo[2,3-c]pyridin-3-yl]-N—[cyclopropyl(2H-1,2,3,4-tetrazol-5-yl)methyl]piperidine-1-carboxamide; trifluoroacetic acid

[chemical structure]

Example 62 (252 mg, 53%) was prepared similarly to Example 61, using cyclopropyl(1H-1,2,3,4-tetrazol-5-yl)methanamine instead of 1H-1,2,3,4-tetrazol-5-ylmethanamine hydrochloride. LCMS (ES⁺): 477.1 [MH]⁺. HPLC: Rt 4.69 min, 99% purity.

### Example 63

### 4-[1-(4-Chlorophenyl)-1H-pyrrolo[2,3-c]pyridin-3-yl]-N-[1-(2H-1,2,3,4-tetrazol-5-yl)cyclobutyl]piperidine-1-carboxamide; trifluoroacetic acid

[chemical structure]

Example 63 (92.0 mg, 19%) was prepared similarly to Example 61, using 1-(1H-1,2,3,4-tetrazol-5-yl)cyclobutan-1-amine instead of 1H-1,2,3,4-tetrazol-5-ylmethanamine hydrochloride. LCMS (ES⁺): 477.1 [MH]⁺. HPLC: Rt 4.57 min, 99% purity.

### Example 64

### 1-(2-{[1-(4-Chlorophenyl)-1H-pyrazolo[3,4-c]pyridin-3-yl]amino}ethyl)imidazolidin-2-one

[chemical structure]

Intermediate 55 (70.0 mg, 0.15 mmol) was dissolved in HCl in EtOH (1.25 M, 20 mL) and stirred for 5 d. The reaction mixture was concentrated in vacuo, partitioned between DCM (15 mL) and sat aq NaHCO₃ (10 mL) and the organic fraction was dried (MgSO₄) and concentrated in vacuo to give the title compound (47.5 mg, 87%) as a white solid. LCMS (ES⁺): 357.1 [MH]⁺. HPLC: Rt 4.89 min, 99.6% purity.

### Biological Tests

### Biological Assays of the SSAO Enzyme Inhibitors

All primary assays were performed at RT. with purified recombinantly expressed human SSAO. Enzyme was prepared essentially as described in Öhman et al. (Protein Expression and Purification 46 (2006) 321-331). In addition, secondary- and selectivity assays were performed using SSAO prepared from various tissues or purified rat recombinant SSAO. The enzyme activity was assayed with benzylamine as substrate by measuring either benzaldehyde production, using ¹⁴C-labeled substrate, or by utilizing the production of hydrogen peroxide in a horseradish peroxidase (HRP) coupled reaction. Briefly, test compounds were dissolved in dimethyl sulfoxide (DMSO) to a concentration of 10 mM. Dose-response measurements were assayed by either creating 1:10 serial dilutions in DMSO to produce a 7 point curve or by making 1:3 serial dilutions in DMSO to produce 11 point curves. The top concentrations were adjusted depending on the potency of the compounds and subsequent dilution in reaction buffer yielded a final DMSO concentration ≦2%.

### Hydrogen Peroxide Detection:.

In a horseradish peroxidase (HRP) coupled reaction, hydrogen peroxide oxidation of 10-acetyl-3,7-dihydroxyphenoxazine produced resorufin, which is a highly fluorescent compound (Zhout and Panchuk-Voloshina. Analytical Biochemistry 253 (1997) 169-174; Amplex® Red Hydrogen Peroxideperoxidase Assay kit, Invitrogen A22188). Enzyme and compounds in 50 mM sodium phosphate, pH 7.4 were set to pre-incubate in flat-bottomed microtiter plates for approximately 15 minutes before initiating the reaction by addition of a mixture of HRP, benzylamine and Amplex reagent. Benzylamine concentration was fixed at a concentration corresponding to the Michaelis constant, determined using standard procedures. Fluorescence intensity was then measured at several time points during 1-2 hours, exciting at 544 nm and reading the emission at 590 nm. For the human SSAO assay final concentrations of the reagents in the assay wells were: SSAO enzyme 1 μg/ml, benzylamine 100 μM, Amplex reagent 20 μM, HRP 0.1 U/mL and varying concentrations of test compound. The inhibition was measured as % decrease of the signal compared to a control without inhibitor (only diluted DMSO). The background signal from a sample containing no SSAO enzyme was subtracted from all data points. Data was fitted to a four parameter logistic model and IC₅₀ values were calculated using the Graph Pad Prism 4 or XLfit 4 programs.

### Aldehyde Detection:

SSAO activity was assayed using 14C-labeled benzylamine and analysed by measuring radioactive benzaldehyde. In a white 96-well optiplate (Packard), 20 μL of diluted test compound was pre-incubated at RT. with 20 μL SSAO enzyme for approximately 15 minutes with continuous agitation. All dilutions were made with PBS. The reaction was initiated by adding 20 μL of the benzylamine substrate solution containing [7-14C] Benzylamine hydrochloride (CFA589, GE Healthcare). The plate was incubated for 1 hour as above after which the reaction was stopped by acidification (10 μL 1 M HCl). Then 90 μL Micro Scint-E solution (Perkin-Elmer) was added to each well and the plate was continuously mixed for 15 minutes. Phase separation occurred instantly and activity was read in a Topcount scintillation counter (Perkin-Elmer). In the final reaction well, the human recombinant SSAO concentration was 10 μg/ml. In order to optimize sensitivity, the substrate concentration was decreased as compared to the HRP coupled assay in order to get a higher fraction of radioactive product. In the human SSAO assay, benzylamine concentration was 40 μM (0.2 μCi/mL). Data was analysed as above.

All of the exemplified compounds of the invention had an IC₅₀ value of 1-2500 nM at SSAO(See Table 7).

TABLE 7
SSAO inhibitory activity
(A: <100 nM, B: 100-500 nM, C: 500-2500 nM)

| Compound | IC₅₀ (nM) |
|---|---|
| 1 | A |
| 2 | B |
| 3 | B |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | B |
| 33 | B |
| 34 | B |
| 35 | B |
| 36 | A |
| 37 | B |
| 38 | B |
| 39 | B |
| 40 | A |
| 41 | B |
| 42 | A |
| 43 | B |
| 44 | C |
| 45 | B |
| 46 | B |
| 47 | B |
| 48 | A |
| 49 | B |
| 50 | B |
| 51 | A |
| 52 | A |
| 53 | C |
| 54 | A |
| 55 | A |
| 56 | A |
| 57 | B |
| 58 | B |
| 59 | A |
| 60 | A |
| 61 | A |
| 62 | A |
| 63 | A |
| 64 | A |

1. A compound of formula (I) or a pharmaceutically acceptable salt, or N-oxide thereof:
R¹—X—R² (I)
wherein
R¹ is phenyl or 6-membered heteroaryl, optionally substituted with one or more substituents selected from halogen, cyano, C₁₋₄-alkyl, halo-C₁₋₄-alkyl, C₁₋₄alkoxy-C₁₋₄alkyl, hydroxy-C₁₋₄-alkyl, cyano-C₁₋₄-alkyl, amino-C₁₋₄-alkyl, C₁₋₄-alkylamino-C₁₋₄-alkyl, di(C₁₋₄-alkyl)amino-C₁₋₄-alkyl, —NR^{4A}R^{4B}, —NR⁶C(O)OR⁵, —NR⁶C(O)R⁵, —NR⁶C(O)NR^{4A}R^{4B}, —C(O)NR^{4A}R^{4B}, —C(O)R⁵, —C(O)OR⁵, and —NR⁶S(O)₂R⁵;
R² is —B-Q-[R³]ₙ or —B—R³;
wherein n=1, 2, 3, or 4
B is a bond, O, NR⁴, —C(O)— or C₁₋₃-alkylene;
Q is saturated or partially unsaturated monocyclic 3-7 membered heterocyclic or C₃₋₄-cycloalkyl ring;
when R² is —B-Q-[R³]ₙ, R³ is independently selected from: 3-7 membered heterocyclyl-, 3-7 membered heterocyclyl-C₁₋₄-alkyl-, (3-7 membered heterocyclyl-C₁₋₄-alkyl)-amino-C₁₋₄-alkyl-, amino-C₁₋₄-alkoxy-C₁₋₄-alkyl-, (amino-C₁₋₄-alkyl)-amino-C₁₋₄-alkyl-, —C₁₋₄-alkyl-NR⁶C(O)OR⁵, —C₁₋₄-alkyl-NR⁶C(O)NR^{4A}R^{4B}, —C₁₋₄-alkyl-C(O)NR^{4A}R^{4B}, (3-7 membered heterocyclyl-C₁₋₄-alkyl)-C(O)—, —C₁₋₄-alkyl-C(O)OR⁵, —OC(O)R⁵, or
—C(O)NR^{9A}R^{9B} wherein R^{9A} and R^{9B} together with the nitrogen to which they are attached form a 3-7 membered cyclic amino group substituted with one or more substituents selected from: C₁₋₄-alkyl, C₁₋₄alkoxy-C₁₋₄alkyl-, C₃₋₇-cycloalkyl, or
—C(O)NR⁶R^{10B} wherein R^{10B} is 3-7 membered heterocyclyl- or 3-7 membered heterocyclyl-C₁₋₄-alkyl-, or —C₁₋₄-alkyl-NR⁶C(O)R⁵; or
when R² is —B—R³, R³ is —NR⁶R^{11B}, wherein R^{11B} is 3-7 membered heterocyclyl-C₁₋₄-alkyl-;
R^{4A}, R^{4B} and R⁵ are each independently selected from hydrogen, C₁₋₄-alkyl-, 3-7 membered heterocyclyl-C₁₋₄-alkyl-, amino-C₁₋₄-alkyl-, 3-7 membered heterocyclyl-, —C₁₋₄-alkyl-NR⁶C(O)OR⁵, C₃₋₇-cycloalkyl,
or R^{4A} and R^{4B} together with the nitrogen to which they are attached form a 3-7 membered cyclic amino group, optionally substituted by one or more substituents selected from: C₁₋₄-alkyl, —NR^{4A}R^{4B};
unless otherwise specified, 3-7 membered heterocyclyl, or the heterocyclyl part of the 3-7 membered heterocyclyl-C₁₋₄-alkyl-, (3-7 membered heterocyclyl-C₁₋₄-alkyl)-amino-C₁₋₄-alkyl-, or (3-7 membered heterocyclyl-C₁₋₄-alkyl)-C(O)— group is optionally substituted with one or more substituents selected from C₁₋₄-alkyl-, —C(O)OR⁵, —C(O)R⁵, —C(O)—NR^{4A}R^{4a}, —NR^{4A}R^{4B}, —C₁₋₄-alkyl-C(O)NR^{4A}R^{4B}, or C₁₋₄alkoxy-C₁₋₄alkyl; and
where present, the diradical —C₁₋₄-alkyl- group directly attached to Q is optionally substituted with one or more groups independently selected from halogen, amino, methoxy, hydroxyl;
R⁴, and R⁶ are each independently selected from hydrogen or C₁₋₄-alkyl; and
X is selected from the radicals of formulae (1-16) wherein the bond marked * is attached to R¹— and the bond marked ** is attached to —R²: [chemical structure] [chemical structure] [chemical structure]
wherein Y is selected from hydrogen, hydroxyl, amino, —NHR⁶, —OCH₃;
Z is selected from hydrogen, fluorine, hydroxyl, C₁₋₄-alkoxy, CONH₂, cyano, SO₂NH₂, amino, —NHR⁶;
W is selected from H, C₁₋₄-alkyl, halo-C₁₋₄-alkyl,
PROVIDED THAT when R² is —B-Q-[R³]ₙ, and R³ is 3-7 membered heterocyclyl-, the heterocyclic ring atom directly bonded to Q is not nitrogen, and
PROVIDED THAT the compound of formula (I) is not:

[chemical structure]

2. The compound according to claim 1 wherein X is selected from the radicals of formulas 1 or 3.

3. The compound according to claim 1 wherein R¹ is phenyl optionally substituted with one or more substituents as defined in claim 1.

4. The compound according to claim 1 wherein R¹ is optionally substituted by halogen, cyano, C₁₋₄-alkyl, halo-C₁₋₄-alkyl.

5. The compound according to claim 1 wherein B is a bond.

6. The compound as claimed in claim 1 wherein R² is —B-Q-[R³]ₙ, and Q is a saturated or partially unsaturated 5 or 6 membered heterocyclic or cycloalkyl ring.

7. The compound as claimed in claim 6 wherein Q is selected from tetrahydrofuranyl, tetrahydropyranyl, piperidinyl, piperazinyl, morpholinyl, cyclohexyl, or any of the foregoing rings comprising a bridge formed by an ethylene or propylene radical.

8. The compound according to claim 1 wherein R² is: [chemical structure]
wherein
T is N or CH
R⁶ is hydrogen or C₁₋₄-alkyl
R^{10B} is 3-7 membered heterocyclyl-, or 3-7 membered heterocyclyl-C₁₋₄-alkyl-, either of which heterocyclic rings is optionally substituted by one or more substituents selected from C₁₋₄-alkyl- and C₁₋₄alkoxy-C₁₋₄alkyl.

9. The compound according to claim 8 wherein R² is [chemical structure]
wherein:
T is N or CH;
P is a direct bond or a diradical selected from methylene, ethylene, or propylene;
R⁶ is hydrogen or C₁₋₄alkyl;
R¹² is selected from hydrogen, C₁₋₄-alkyl- and C₁₋₄alkoxy-C₁₋₄alkyl-.

10. The compound according to claim 8 wherein R² is: [chemical structure]
wherein
T is N or CH;
P is a diradical selected from methylene, ethylene, or propylene;
R⁶ is hydrogen or C₁₋₄-alkyl;
R¹² is selected from hydrogen, C₁₋₄-alkyl, and C₁₋₄alkoxy-C₁₋₄alkyl-.

11. The compound according to claim 1 wherein R² is: [chemical structure]
wherein
R³ is —C₁₋₄-alkylC(O)NR^{4A}R^{4B} wherein
R^{4A} and R^{4B} are each independently selected from hydrogen, C₁₋₄-alkyl-, and amino-C₁₋₄-alkyl-, or R^{4A} and R^{4B} together with the nitrogen to which they are attached form a 3-7 membered cyclic amino group, optionally substituted by one or more substituents selected from: C₁₋₄-alkyl, or —NR^{4A}R^{4B}.

12. The compound as claimed in claim 1 which is:
4-[1-(4-Chlorophenyl)-1H-pyrrolo[2,3-c]pyridin-3-yl]-N-(piperidin-4-ylmethyl)piperidine-1-carboxamide;
4-[1-(4-Chlorophenyl)-1H-pyrrolo[2,3-c]pyridin-3-yl]-N-(1-methylpiperidin-4-yl)piperidine-1-carboxamide;
4-[1-(4-Chlorophenyl)-1H-pyrrolo[2,3-c]pyridin-3-yl]-N-[(1-methylpiperidin-4-yl)methyl]piperidine-1-carboxamide;
4-[1-(4-Chlorophenyl)-1H-pyrrolo[2,3-c]pyridin-3-yl]-N-[(1-ethylpiperidin-4-yl)methyl]piperidine-1-carboxamide;
4-[1-(4-Chlorophenyl)-1H-pyrrolo[2,3-c]pyridin-3-yl]-N-methyl-N-[(1-methylpiperidin-4-yl)methyl]piperidine-1-carboxamide;
4-[1-(4-Chlorophenyl)-1H-pyrrolo[2,3-c]pyridin-3-yl]-N-[2-(piperazin-1-yl)ethyl]piperidine-1-carboxamide;
4-[1-(4-Chlorophenyl)-1H-pyrrolo[2,3-c]pyridin-3-yl]-N-[2-(1-methylpiperidin-4-yl)ethyl]piperidine-1-carboxamide;
4-[1-(4-Chlorophenyl)-1H-pyrrolo[2,3-c]pyridin-3-yl]-N-[3-(morpholin-4-yl)propyl]piperidine-1-carboxamide;
4-[1-(4-Chlorophenyl)-1H-pyrrolo[2,3-c]pyridin-3-yl]-N-{[1-(propan-2-yl)piperidin-4-yl]methyl}piperidine-1-carboxamide;
4-[1-(4-Chlorophenyl)-1H-pyrrolo[2,3-c]pyridin-3-yl]-N-{[1-(2-methoxyethyl)piperidin-4-yl]methyl}piperidine-1-carboxamide;
4-[1-(4-Chlorophenyl)-1H-pyrazolo[3,4-c]pyridin-3-yl]-N-[(1-methylpiperidin-4-yl)methyl]piperazine-1-carboxamide;
N-(2-Aminoethyl)-2-{4-[1-(4-chlorophenyl)-1H-pyrazolo[3,4-c]pyridin-3-yl]morpholin-3-yl}acetamide;
2-{4-[1-(4-Chlorophenyl)-1H-pyrazolo[3,4-c]pyridin-3-yl]morpholin-3-yl}-1-[(3S)-3-(dimethylamino)pyrrolidin-1-yl]ethan-1-one;
or a pharmaceutically acceptable salt, or N-oxide thereof.

13. A pharmaceutical composition comprising compound as claimed in claim 1, together with one or more pharmaceutically acceptable carriers and/or excipients.

14-19. (canceled)

20. A method for the treatment of inflammation, an inflammatory disease, an immune or an autoimmune disorder, or inhibition of tumor growth, which comprises administering to a subject suffering such disease an effective amount of a compound as claimed in claim 1.

21. The method of treatment of claim 20, wherein the inflammatory disease is rheumatoid arthritis, chronic obstructive pulmonary disease or atopic dermatitis.

22. The method of treatment of claim 20, for inhibition of tumor growth.

23. The method of treatment of claim 20, wherein the inflammation or inflammatory disease or immune or autoimmune disorder is arthritis, synovitis, vasculitis, a condition associated with inflammation of the bowel, atherosclerosis, multiple sclerosis, Alzheimer's disease, vascular dementia, a pulmonary inflammatory disease, a fibrotic disease, an inflammatory disease of the skin, systemic inflammatory response syndrome, sepsis, an inflammatory and/or autoimmune condition of the liver, type I or type II diabetes and/or the complications thereof, chronic heart failure, congestive heart failure, an ischemic disease, or myocardial infarction and/or the complications thereof.

24. The method of treatment of claim 23, wherein the arthritis is rheumatoid arthritis, juvenile rheumatoid arthritis, osteoarthritis or psoriatic arthritis.

25. The method of treatment of claim 23, wherein the condition associated with inflammation of the bowel is Crohn's disease, ulcerative colitis, inflammatory bowel disease or irritable bowel syndrome.

26. The method of treatment of claim 23, Wherein the pulmonary inflammatory disease is asthma, chronic obstructive pulmonary disease, or acute respiratory distress syndrome.

27. The method of treatment of claim 23, wherein the fibrotic disease is idiopathic pulmonary fibrosis, cardiac fibrosis or systemic sclerosis (scleroderma).

28. The method of treatment of claim 23, wherein the inflammatory disease of the skin is contact dermatitis, atopic dermatitis or psoriasis.

29. The method of treatment of claim 23, wherein the inflammatory and/or autoimmune condition of the liver is autoimmune hepatitis, primary biliary cirrhosis, alcoholic liver disease, sclerosing cholangitis, or autoimmune cholangitis.
